(19) United States
(12) Patent Application Publication
(10) Pub. No.: US 2007/0255176 A1
(43) Pub. Date: Nov. 1, 2007

(54) Voiding detection with learning mode

(75) Inventors: John C. Rondoni, Plymouth, MN (US); Martin T. Gerber, Maple Grove, MN (US)

Correspondence Address: SHUMAKER & SIEFFERT, P. A., 1625 RADIO DRIVE, SUITE 300, WOODBURY, MN, 55125, US

(73) Assignee: Medtronic, Inc.

(21) Appl. No.: 11/414,504

(22) Filed: Apr. 28, 2006

(51) Int. Cl. A61B 5/00

(52) U.S. Cl. 600/573

(57) ABSTRACT

The disclosure describes an implantable stimulation system that learns to identify a voiding signature of a bladder and log the voiding events. The system may adjust stimulation therapy according to the voiding signature. The system includes an implantable neurostimulator and a sensor that senses a physiological event indicative of a voiding event. The sensor may sense neurological activity, bladder dimensions, bladder characterizes, external wetness, or other activities related to patient voiding. The neurostimulation correlates the sensed event to an input by a user to learn what sensed data is indicative of a voiding event. In addition, the system may use a secondary sensor to negate a voiding signature detection when the patient may not be having a voiding event.

### TECHNICAL SUPPORT

The invention relates to implantable medical devices and, more particularly, implantable sensors.

### BACKGROUND

Urinary incontinence, or an inability to control urinary function, is a common problem afflicting people of all ages, genders, and races. Various muscles, nerves, organs and conduits within the urinary tract cooperate to collect, store and release urine. A variety of disorders may compromise urinary tract performance and contribute to incontinence. Many of the disorders may be associated with aging, injury or illness.

In some cases, urinary incontinence can be attributed to improper sphincter function, either in the internal urinary sphincter or external urinary sphincter. For example, aging can often result in weakened sphincter muscles, which causes incontinence. Some patients also may suffer from nerve disorders that prevent proper triggering and operation of the bladder or sphincter muscles. Nerves running though the pelvic floor stimulate contractility in the sphincter. A breakdown in communication between the nervous system and the urinary sphincter can result in urinary incontinence.

Electrical stimulation of nerves in the pelvic floor may provide an effective therapy for a variety of disorders, including urinary incontinence. For example, an implantable electrical stimulator may be provided. The electrical stimulator may be a neurostimulator that delivers electrical stimulation to the sacral nerve to induce sphincter constriction and thereby close or maintain closure of the urethra at the bladder neck. In addition, electrical stimulation of the bladder wall may enhance pelvic floor muscle tone and assist fluid retention in the bladder or voiding fluid from the bladder. An appropriate course of neurostimulation therapy may be aided by a sensor that monitors physiological conditions of the bladder. In some cases, an implantable stimulation device may deliver stimulation therapy based on the level or state of a sensed physiological condition.

### SUMMARY

The invention is directed to an implantable stimulation system that detects a bladder voiding event based on a voiding signature that is characteristic of an actual voiding event observed for a patient. The system may include an implantable neurostimulator and a sensor that senses a physiological condition indicative of a voiding event. The system may adjust electrical stimulation therapy according to the indication of a voiding event detected and/or log the voiding event for analysis. For example, upon detecting an involuntary voiding event, the stimulation system may apply stimulation to halt the voiding event.

The sensor may sense neurological activity, bladder dimensions, bladder characteristics, external wetness, or other characteristics related to urinary voiding. The voiding signature may specify one or more characteristics of the sensor signal that indicate the actual voiding event. By comparing the sensor signal to the voiding signature, the system can more accurately detect a voiding event. In addition, a secondary sensor may be used for cross-correlation to prevent false detection of voiding event.

The voiding signature may be defined, for example, based on one or more characteristics of the sensor signal observed during a natural voiding event by the patient, or based on one or more characteristics of the sensor signal observed during an induced voiding event by the patient. Voiding may be induced, for example, by filling the bladder with fluid via a catheter. In either case, the voiding signature is obtained for the particular patient, facilitating patient-individualized voiding detection.

In one embodiment, the invention provides a method comprising receiving a sensor signal, comparing the sensor signal to a voiding signature indicating an actual urinary voiding event in a patient, and indicating a voiding event based on the comparison.

In another embodiment, the invention provides a system comprising a sensor that generates a sensor signal, and a processor that compares the sensor signal to a voiding signature indicating an actual urinary voiding event in a patient, and indicates a voiding event based on the comparison.

In an additional embodiment, the invention provides a computer-readable medium comprising instructions to cause a processor to receive a sensor signal, compare the sensor signal to a voiding signature indicating an actual urinary voiding event in a patient, and indicate a voiding event based on the comparison.

In various embodiments, the invention may provide one or more advantages. For example, correlating the sensor data with a known voiding event for a patient allows the system to use a voiding signature to more accurately identify a voiding event. In this manner, the system can more reliably identify a voiding event for purposes of modifying stimulation therapy and/or logging voiding events. The system may continue to learn throughout the therapy by continually monitoring feedback data. In addition, the stimulation system may detect a voiding event even before the event actually occurs, which may increase therapeutic efficacy.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating an implantable stimulation system, incorporating a nerve sensor that senses bladder events.

FIG. 2 is a schematic diagram illustrating an implantable stimulation system, incorporating an external bladder sensor that senses bladder events.

FIG. 3 is a schematic diagram illustrating an implantable stimulation system, incorporating an internal bladder sensor that senses bladder events.

FIG. 4 is a schematic diagram illustrating an implantable stimulation system, incorporating a sphincter force sensor that senses bladder events.

FIG. 5 is a schematic diagram illustrating an implantable stimulation system, incorporating a wearable sensor that senses bladder events.

FIG. 6 is a schematic diagram illustrating an implantable stimulation system, incorporating a wearable wetting sensor that senses bladder events.

FIG. 7 is a cross-sectional side view of an implantable sensor placed within a tissue of a patient.

FIG. 8 is a schematic diagram illustrating endoscopic deployment of the implantable sensor of FIG. 7.

FIG. 9 is a cross-sectional side view of an implantable sensor attached to a tissue of a patient.

FIG. 10 is a bottom view of the implantable sensor of FIG. 9.

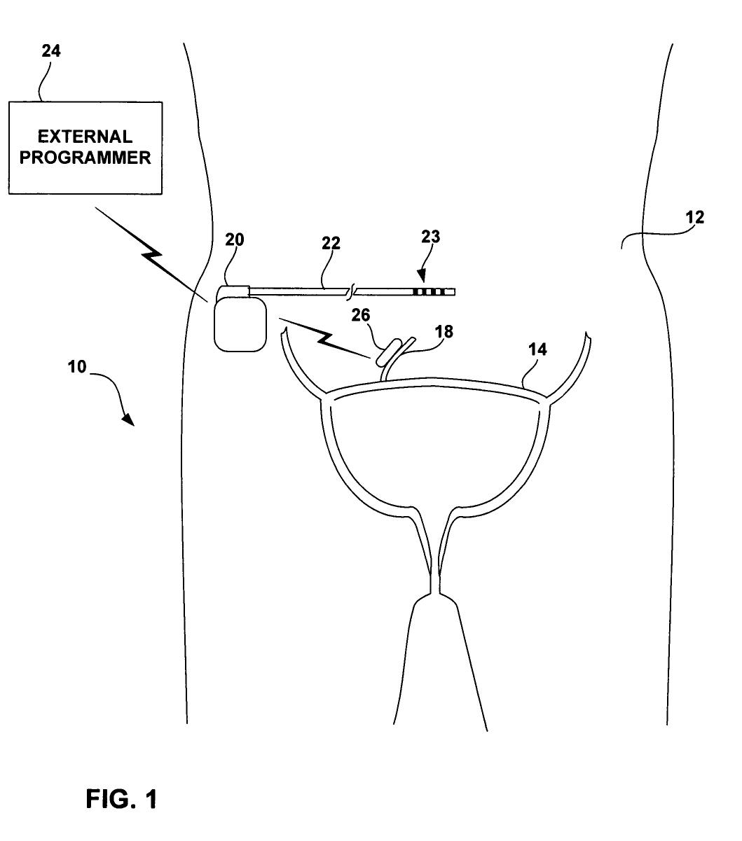

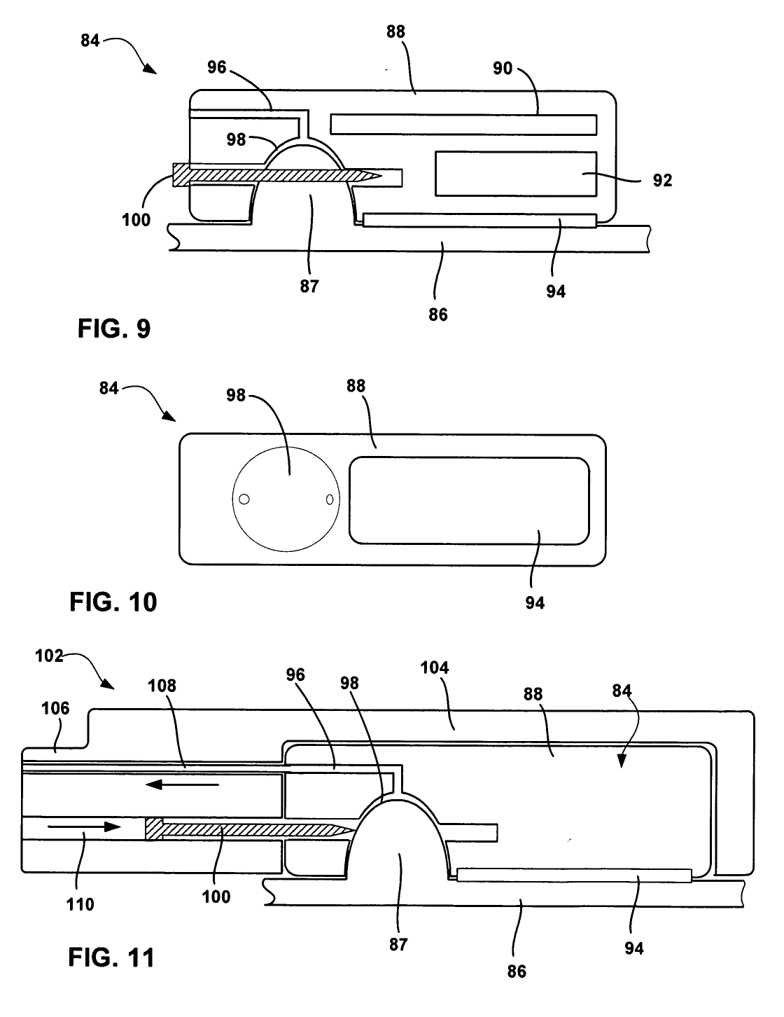

FIG. 1 is a cross-sectional side view of a deployment device during deployment and fixation of the implantable sensor of FIG. 9

FIG. 12 is an enlarged schematic diagram illustrating an implantable sensor sutured to a tissue of a patient.

FIG. 13 is an enlarged, bottom view of the implantable sensor of FIG. 12.

FIG. 14 is a functional block diagram illustrating various components of an exemplary implantable sensor.

FIG. 15 is a functional block diagram illustrating various components of an implantable stimulator that communicates wirelessly with an implantable sensor.

FIG. 16 is a flow chart illustrating a technique for teaching an implantable medical device to identify bladder voiding events

FIG. 17 is a flow chart illustrating a technique for teaching an implantable medical device to identify bladder voiding events from a sensor in a clinic before therapy begins.

FIG. 18 is a flow chart illustrating a technique for teaching an implantable medical device to identify bladder voiding events from a sensor during patient therapy.

FIG. 19 is a flow chart illustrating a technique for teaching an implantable medical device to identify bladder voiding events from multiple sensors during patient therapy.

FIG. 20 is a graph illustrating definition of a voiding signature as a classification map in a three-dimensional space defined by three different sensor signal values.

### DETAILED DESCRIPTION

Urinary incontinence is a condition that affects the quality of life and health of many people. Tracking urinary voiding events may be important in quantifying the number of events a patient has every day or qualifying the severity of the urinary incontinence condition. A sensor, which may be implanted, may sense physiological events occurring within the patient. However, the sensing data generated by the sensor needs to be interpreted to identify a voiding event that needs to be logged. In accordance with this disclosure, a sensor device employs a learning mode to correlate sensing data with an actual voiding event for a patient. In this manner, the sensor device establishes a voiding signature that can be used to distinguish sensed physiological events correlating with a voiding event from those that do not correlate with a voiding event.

Incontinence may be treated with electrical stimulation therapy that prevents urine from leaving the bladder when a patient does not wish to void urine. In combination with tracking voiding events, the electrical stimulation may be delivered to nerves, i.e. sacral or pudendal nerves, or directly to a urinary sphincter, where the stimulation causes the urinary sphincter to constrict and retain urine within the bladder. Electrical stimulation may also be directed to other muscles of the pelvic floor because some of these muscles play a role in controlling urinary voiding events.

The feedback from a sensor device that senses physiological events may aid in timing the electrical stimulation to coincide with the onset of bladder voiding events and non-voiding events. For example, a sensor may detect a voiding signature indicating that the patient is voluntarily attempting to void the bladder. In this case, the stimulator may reduce stimulation therapy in response to the sensed voiding signature to allow the patient to urinate. Alternatively, the sensor may detect that involuntary voiding is occurring. In this case, the stimulator may respond by increasing stimulation intensity to prevent voiding. In each case, the neurostimulator may communicate with a sensor device that correctly identifies voiding events to more effectively treat the patient. In some embodiments, a sensor may sense physiological events that indicate voiding is not occurring. This type of sensor may aid the system in learning to reduce the number of false positive voiding events detected by one or more other sensors.

In addition, the data may be stored in a voiding log that is more accurate due to the voiding signature. The voiding events may be correlated with other patient events to create a more precise voiding signature, where a data logger stores the voiding log according to the voiding signature. A clinician may analyze the voiding log or the programmer may automatically review the voiding log to adjust one or more stimulation parameters of the stimulation therapy. This analysis may be done at pre-defined periodic intervals or at the request of the patient of the clinician.

As described herein, a voiding event generally refers to an actual attempt to void urine from the bladder by a patient. The voiding event may be natural or induced. In particular, a natural voiding event occurs according to the natural body function of the patient. An induced voiding event occurs when fluid is added to the bladder using a catheter to induce a voiding event and thereby simulate natural voiding. An implantable stimulation system, as described herein, makes use of correlation of one or more sensor signals with one or more voiding signatures to identify a natural voiding event, and take specified action such as controlling stimulation or modifying stimulation parameters, or simply logging the detected voiding event. Natural and induced voiding events may be used to define a voiding signature for a particular patient so that natural voiding events can be more reliably identified during normal operation of the system.

FIG. 1 is a schematic diagram illustrating an implantable stimulation system, incorporating a nerve sensor that senses bladder events. As shown in FIG. 1, system 10 includes an implantable nerve sensor 26, implantable neurostimulator 20 and external programmer 24 shown in conjunction with a patient 12. Nerve sensor 26 may sense neuronal activity associated with changes in bladder states. Changes in bladder states may include changes in bladder size, bladder wall thickness, shape, volume, muscle activity or sphincter activity associated with bladder 14. In the example of FIG. 1, nerve sensor 26 is wireless and is implanted adjacent to a nerve that innervates bladder 14 or a related tissue involved with urination. Nerve sensor 26 is in wireless communication with neurostimulator 20, and the neurostimulator is in wireless communication with external programmer 24.

Nerve sensor 26 is implanted adjacent to sacral nerve 18 in the example of FIG. 1. Sacral nerve 18 exits from the patient's sacrum (not shown). The sacrum is the inferior section of the spinal cord, and many nerves leave the sacrum toward other body tissues. These nerves include sacral nerve 18, the pudendal nerve, the sacral plexus, and many other nerves. Some of these nerves innervate bladder 14, the urinary sphincter (not shown), and muscles of the pelvic floor (not shown). While nerve sensor 26 is implanted adjacent to sacral nerve 18, other embodiments may include the nerve sensor implanted adjacent to other nerves of patient 12. However, nerve sensor 26 may also be placed adjacent to many other nerves, such as the pudendal nerve or within the sacral plexus. In other embodiments, nerve sensor 26 may include a cuff electrode, paddle electrode, a flexible electrode, or a ring electrode.

Nerve sensor 26 is shown as a wireless sensor, and the nerve sensor may communicate with external programmer 24 instead of or in addition to neurostimulator 20. In other embodiments, the nerve sensor 26 may be wired via a lead connected to neurostimulator 20. In other embodiments, nerve sensor 26 may transmit data via wired or wireless communication to a data logger implanted in or located external of patient 12 instead of neurostimulator 20. In this case, voiding events may not be used to control stimulation, but to diagnose or evaluate patient condition.

Nerve impulses traveling through sacral nerve 18 are detected by nerve sensor 26 through one or more electrodes or chemical sensors. Many nerve signals are transmitted through sacral nerve 18. The variety of nerve signals and the variation of device placement with respect to the nerve may significantly change any detected signals from the signals expected from the nerve. For this reason, processing circuitry associated with nerve sensor 26 is configured to learn which signals indicate that a voiding event will occur or is occurring, i.e., a voiding signature. This sensor learning may occur during a normal daily routine of patient 12 or in a clinic. In a normal daily routine, patient 12 may use external programmer 24 to log a voiding event when it occurs, and the data acquired by nerve sensor 26 is correlated with the logged voiding event to define the voiding signature of bladder 14. This correlation will train neurostimulator 20, external programmer 24, or system 10 to identify the voiding signature detected with nerve sensor 26.

Similarly, data from nerve sensor 26 may be correlated to voiding events in a clinic setting, except that bladder 14 may be directly filled via a catheter to cause a voiding event in a shorter amount of time. Filling the bladder 14 may be viewed as inducing or simulating a voiding event in the patient. Voiding events that occur in the ordinary function of the patient's body can be viewed as natural voiding events. Neurostimulator 20 or external programmer 24 may also identify data of nerve sensor 26 that corresponds to a full bladder 14 and may increase stimulation therapy to retain urine. Neurostimulator 20 or external programmer 24 may continue to correlate nerve sensor 26 data with voiding events throughout stimulation therapy to further refine the voiding signature and adapt the voiding signature to changes in the physiology or anatomy of the patient. Establishing a voiding signature may assist neurostimulator 20 and/or nerve sensor 26 in identifying voiding events for individual patients on a customized basis. In this manner, by customizing voiding detection for each patient, voiding event sensing can be made more robust across a potentially diverse population of patients.

System 10 allows neurostimulator 20 to recognize multiple states of bladder 14. Data from nerve sensor 26 may be graphed, placed in a table, or used to generate equations that fully characterize the function of bladder 14. The voiding signature includes recognition of various bladder states. For example, multiple voiding signatures may be established for different stages of bladder operation. The voiding signature may be determined by neurostimulator 20 or external programmer 24. In some embodiments, raw data logged in neurostimulator 20 is transmitted to external programmer 24 for processing and determining the voiding signature. The voiding signature may then be transmitted back to neurostimulator 20 to continue effective stimulation therapy. Processing data in external programmer 24 may reduce battery consumption in neurostimulator 20 and eliminate the need for a processor within the neurostimulator to control processes other than the stimulation therapy.

Neurostimulator 20 at least partially prevents unwanted urinary voiding events by stimulating a pelvic floor nerve, a pelvic floor muscle, or the urinary sphincter. Neurostimulator 20 includes a pulse generator that generates electrical pulses and delivers the electrical pulses to a target tissue, e.g., the urinary sphincter, via lead 22 and one or more electrodes 23 located at the distal end of the lead. Neurostimulator 20 may utilize the urinary voiding events detected by nerve sensor 26 to adjust one or more stimulation parameters when a voiding event is detected. For example, neurostimulation 20 may adjust voltage or current amplitude, pulse width or pulse frequency. By correlating sensed physiological data with an actual voiding event, nerve sensor 26 and neurostimulator 20 learn how to identify a voiding event and can take action when one is detected.

In some embodiments, system 10 may include a negative feedback mechanism to indicate when a voiding event is not occurring. This mechanism may be useful in indicating false positive data from nerve sensor 26. For example, the negative feedback mechanism may be an accelerometer that indicates when patient 12 is moving. When patient 12 moves, nerve signals transmitted on sacral nerve 18 may mimic the voiding signature because pelvic floor muscles are contracted. In this example, accelerometer data is used as a cross-correlation with data from sensor 26 so that system 10 does not mistakenly detect a voiding event when the voiding signature is detected.

System 10 is directed to urinary voiding detection with a voiding signature, but other types of physiological functions may be detected and used to learn when other dysfunctions occur. For example, system 10 may be used to treat sexual dysfunction, fecal incontinence, gastro-intestinal disorders, or chronic pain. Multiple sensors may be capable of detecting multiple dysfunctions and system 10 may treat more than one physiological function at one time. However, application of system 10 to urinary incontinence will be described for purposes of illustration.

Other types of sensors, in addition or as an alternative to nerve sensors, may be used to detect changes in voiding activity. Examples of other sensors include pressure sensors, deformation sensors, ultrasound sensors, pH sensors, wetness sensors, sound sensors, size sensors, or any other sensor that may detect a change in fluid flow, fluid volume, or bladder changes. These sensors may be modified to be placed in multiple locations of patient 12. For example, a piezoelectric sensor may be placed adjacent to the urinary sphincter to detect muscle movement of the sphincter that indicates a voiding event is occurring.

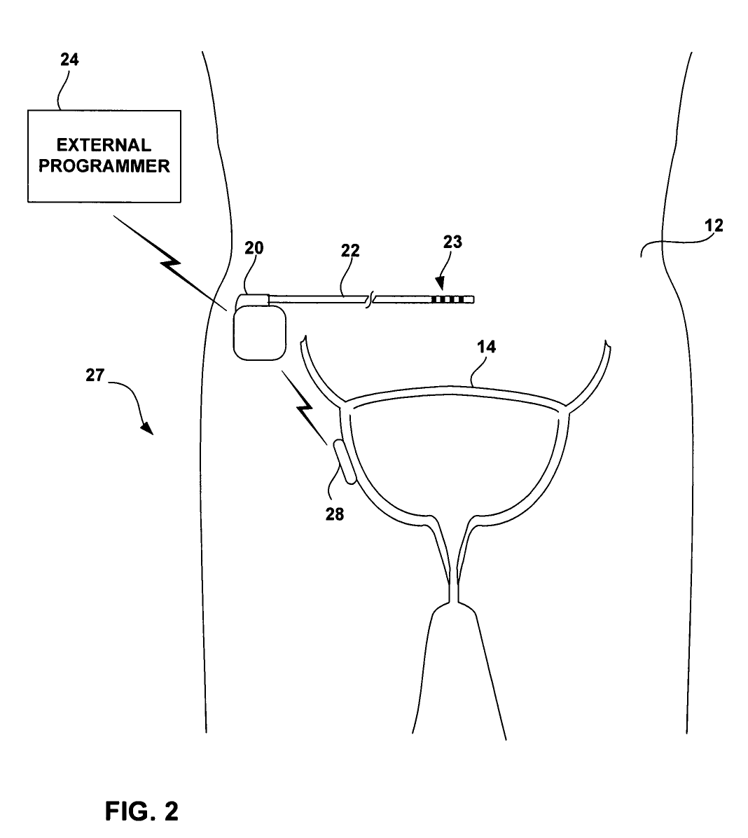

Some examples of sensors are described in more detail in FIGS. 2-6. Several exemplary sensors are also described in the following references: U.S. patent application Ser. No. 11/194,076, entitled “EXTERNAL BLADDER SENSOR FOR SENSING BLADDER CONDITION,” filed on Jul. 29, 2005; U.S. patent application Ser. No. 11/193,310, entitled “TRANSMEMBRANE SENSING DEVICE FOR SENSING BLADDER CONDITION,” filed on Jul. 29, 2005; U.S. patent application Ser. No. 11/116,952, entitled “FLEXIBLE TUBE SENSOR FOR SENSING URINARY SPHINCTER PRESSURE,” filed on Apr. 28, 2005; U.S. patent application Ser. No. 11/117,064, entitled “IMPLANTABLE OPTICAL PRESSURE SENSOR FOR SENSING URINARY SPHINCTER PRESSURE,” filed on Apr. 28, 2005; U.S. patent application Ser. No. 11/117,079, entitled “MULTI-TUBE SENSOR FOR SENSING URINARY SPHINCTER AND URETHRAL PRESSURE,” filed on Apr. 28, 2005; U.S. patent application Ser. No. 11/261,443, entitled “IMPEDANCE-BASED BLADDER SENSING,” filed on Oct. 28, 2005, the entire content of each of which is hereby incorporated by reference.

FIG. 2 is a schematic diagram illustrating an implantable stimulation system, incorporating an external bladder sensor that senses bladder events. As shown in FIG. 2, system 27 incorporates bladder sensor 28 instead of nerve sensor 26 of FIG. 1. Bladder sensor 28 is located on the exterior of bladder 14 and wirelessly communicates with neurostimulator 20. Neurostimulator 20 delivers stimulation therapy to treat urinary incontinence and may also wirelessly communicate with external programmer 24. Bladder sensor 28 may be implanted at an external surface of bladder 14, e.g., with sutures or another fixation mechanism, to monitor changes in bladder dimensions or activity in the bladder wall muscle, such as contractile activity.

Deformation of bladder 14 is detected by a sensing element such as strain gauge, for example, in bladder sensor 28 to generate information regarding the amount of urine in bladder 14, i.e., a fill stage, or the occurrence of bladder contraction above a threshold. A fill stage or bladder contraction may be considered bladder activity or bladder condition information that can be used to determine a voiding signature. The detected bladder activity is correlated to input from patient 12 identifying a voiding event to determine the voiding signature. The learning mode for determining the voiding signature may continue during chronic stimulation, similar to that of system 10 of FIG. 1.

Neurostimulator 20 may activate or adjust stimulation in response to the voiding signature detected by bladder sensor 28. Bladder sensor 28 transmits the sensed bladder data to at least one of neurostimulator 20 or external programmer 24 by wireless telemetry. External programmer 24 may process the data and transmit the a signal indicative of detection of the voiding signature to neurostimulator 20 for use during stimulation therapy. The bladder data may be transmitted to neurostimulator 20 as individual measurement samples, or pre-processed bladder condition information based on one or more measurement samples, or the information may be transmitted only when a significant change is detected.

Neurostimulator 20 or external programmer 24 may record data, generate adjustments to electrical stimulation if a voiding signature is detected, or both. In some embodiments, bladder sensor 28 may support purely diagnostic purposes, such as urodynamic study, e.g., by transmission of data to external programmer 24. The voiding signature may be used during a different therapy or in deciding if stimulation therapy is appropriate. In other embodiments, bladder sensor 28 may form part of a closed loop feedback system for delivery and adjustment of neurostimulation therapy by neurostimulator 20 to patient 12.

In some embodiments, bladder sensor 28 may be wired via a lead connected to neurostimulator 20. In other embodiments, bladder sensor 28 may transmit data via wired or wireless communication to a data logger implanted in or located external of patient 12 instead of neurostimulator 20. In this case, voiding events may not be used to control stimulation, but to diagnose or evaluate patient 12 condition. In alternative embodiments, bladder sensor 28 may communicate with both a neurostimulator and an implanted or external data logger.

In some embodiments, similar to system 10, system 27 may include a negative feedback mechanism to indicate when a voiding signature is incorrectly detected. This mechanism may be useful in indicating false positive data from bladder sensor 28. For example, the negative feedback mechanism may be an accelerometer that indicates when patient 12 is moving. When patient 12 moves, bladder 14 may change shape and mimic the voiding signature associated with a certain bladder dimension. In this manner, system 10 does not misinterpret the voiding signature.

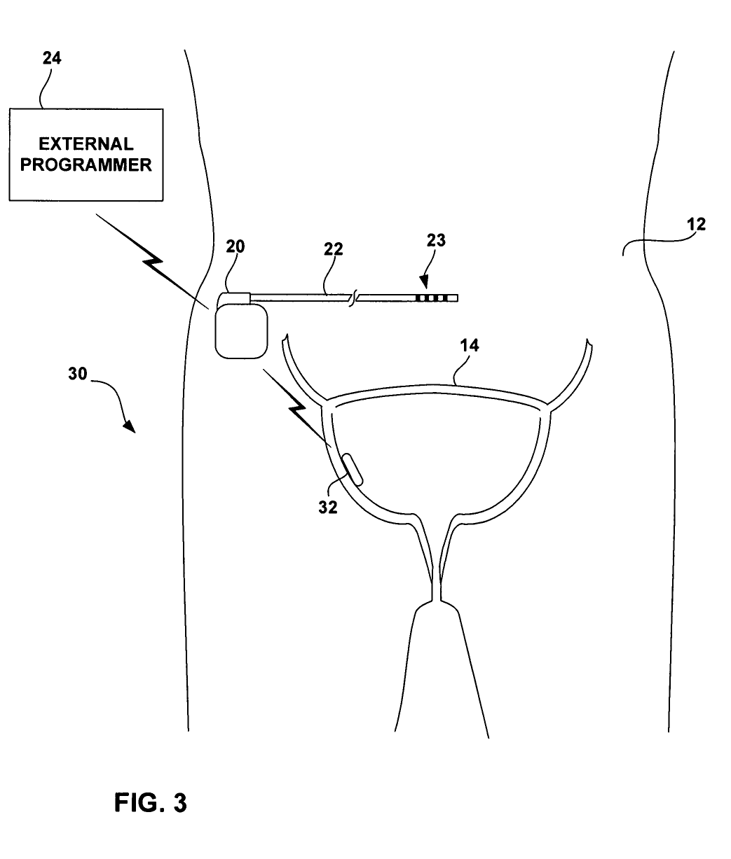

FIG. 3 is a schematic diagram illustrating an implantable stimulation system, incorporating an internal bladder sensor that senses bladder events. As shown in FIG. 3, system 30 incorporates an internal bladder sensor 32 instead of nerve sensor 26 of FIG. 1 or external bladder sensor 28 of FIG. 2. Bladder sensor 32 is located on the inside of bladder 14 and wirelessly communicates with neurostimulator 20. Neurostimulator 20 delivers stimulation therapy to treat urinary incontinence and may also wirelessly communicate with external programmer 24.

Bladder sensor 32 is implanted at an internal surface of the wall of bladder 14 to monitor changes in bladder dimensions, bladder volume, urine ion concentrations, bladder wall muscle activity, or other physiological conditions. In contrast to bladder sensor 28, bladder sensor 32 may provide more information regarding the interior of bladder 14. Deformation of bladder 14 is detected by a sensing element such as strain gauge, for example, in bladder sensor 32 to generate information regarding the amount of urine in bladder 14, i.e., a fill stage, or the occurrence of bladder contraction above a threshold.

Alternatively, bladder sensor 28 may detect electrical impedance to measure a volume of bladder 14, use ultrasound to measure the distance across the bladder, or measure a pH of the urine to indicate a voiding signature. A fill stage or bladder contraction may be considered bladder activity or bladder condition information that can be used to determine the voiding signature. The detected bladder activity is correlated to patient 12 input identifying a voiding event to determine the voiding signature. The learning mode for determining the voiding signature may continue during chronic stimulation, similar to that of system 10 of FIG. 1.

Sensor 32 may be anchored to the inside wall of bladder 14 with any of a variety of fixation mechanisms. System 30 may operate in a manner very similar to the systems 10 and 27, previously described. All previously described functions are also applicable with regard to bladder sensor 32. In alternative embodiments, two or more sensors 26, 28, or 32 may be used to collect a greater amount of data to precisely define the voiding signature. Other sensors still to be described below may also be used.

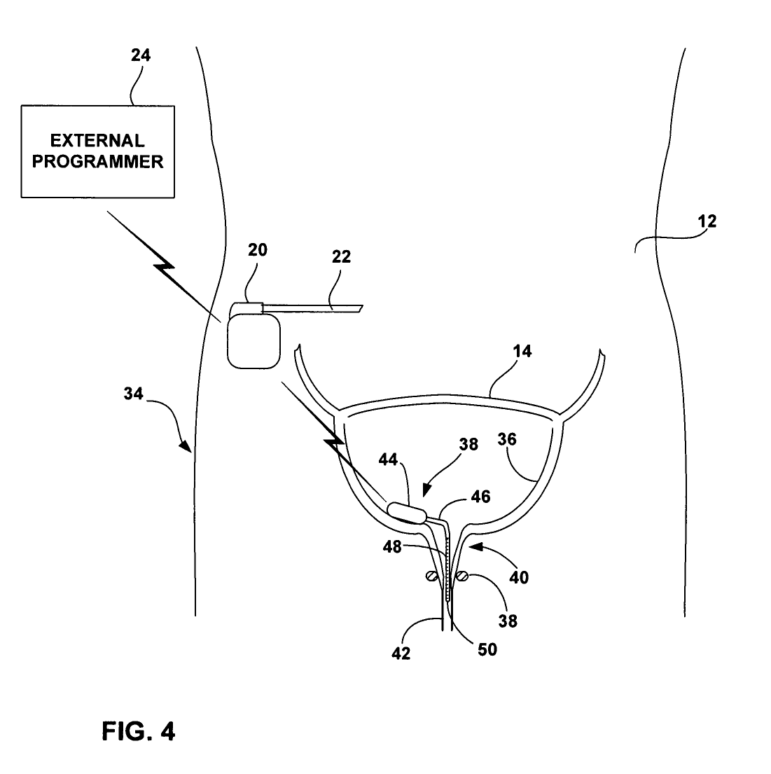

FIG. 4 is a schematic diagram illustrating an implantable stimulation system, incorporating a sphincter force sensor that senses bladder events. As shown in FIG. 4, system 34 includes external programmer 24, neurostimulator 20, and pressure sensor 38 attached to bladder wall 26. Pressure sensor 38 includes housing 44, fluid tube 46, fluid 48 and cap 50 to measure the pressure of urinary sphincter 38. Fluid tube 46 resides within bladder neck 40 and urethra 42. As sphincter 38 constricts to prevent urine from leaking out of bladder 14, pressure sensor 38 measures high pressure. A measured pressure drop may indicate that urine is being voided, or will be voided, from bladder 14.

Pressure sensor 38 directly measures the closing pressure of urinary sphincter 38 to monitor voiding events. Patient 12 may enable system 34 learn what the voiding signature is by indicating to external programmer 24 when urine has voided from bladder 14. Pressure sensor 30 wirelessly communicates with neurostimulator 20 to transmit pressure data to the neurostimulator, at which time the data may be sent to external programmer 24 for correlation and determination of the voiding signature. In some embodiments, pressure sensor 38 directly communicates with external programmer 24. System 34 may continually learn to define the voiding signature by continuing to correlate the voiding events with the pressure data generated by pressure sensor 38.

In the example of FIG. 4, pressure sensor 38 is a mechanical pressure sensor that uses fluid 48 within a fluid tube 46 to transfer pressure from sphincter 38 to a strain gauge (not shown) within housing 44. When sphincter 38 squeezes to prevent urine from leaking into urethra 42, the strain gauge of pressure sensor 38 deflects and generates an electrical signal that is transformed into digital data representative of the pressure of sphincter 38. Pressure sensor 38 does not interfere with normal urological function or stimulation therapy. Pressure sensor 38 allows neurostimulator 20 to identify voiding signatures and respond with stimulation accordingly.

System 34 functions in a manner similar to systems 10, 27, and 30, but differs in the type of sensor used to detect voiding events. Pressure sensor 38 may remain within bladder 14 for an extended period of time to provide steady data. Pressure sensor 30 may be implanted through urethra 42 to eliminate the need for surgical implantation. When patient 12 no longer needs pressure sensor 38, the clinician may remove a pin and the patient may pass the sensor out of bladder 14 with a voiding event.

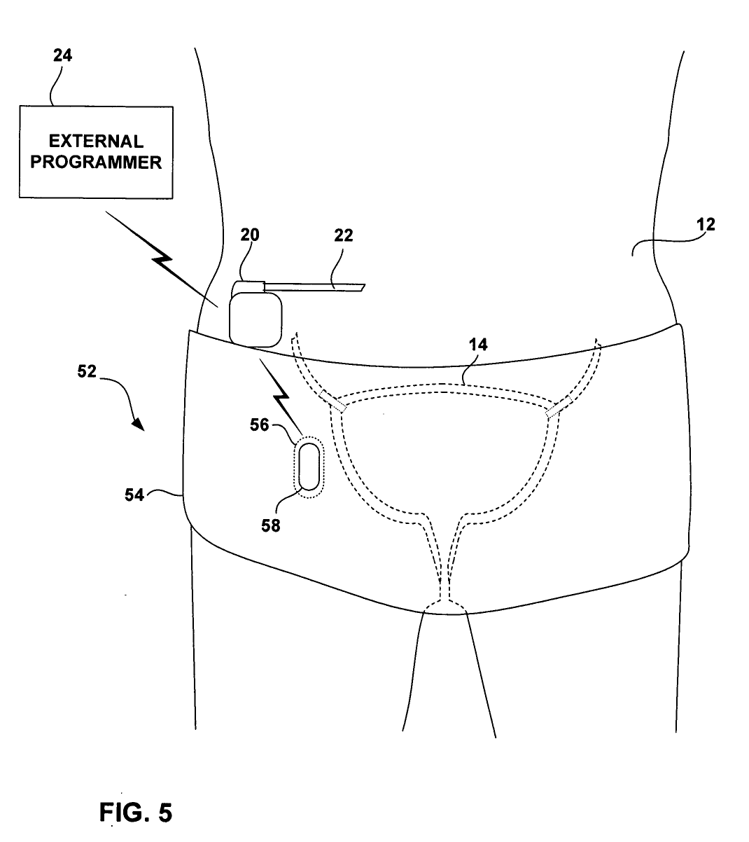

FIG. 5 is a schematic diagram illustrating an implantable stimulation system, incorporating a wearable sensor that senses bladder events. As shown in FIG. 5, system 52 includes undergarment 54, neurostimulator 20, and external programmer 24. Undergarment 54 includes pocket 56 that secures ultrasound sensor 58. System 52 operates similar to system 10 to learn to detect a voiding signature, but ultrasound sensor 58 is located external to patient 12. Ultrasound sensor 58 wirelessly communicates with neurostimulator 20, and neurostimulator 20 communicates with external programmer 24. In some embodiments, ultrasound sensor 58 may directly communicate with external programmer 24.

Ultrasound sensor 58 is placed close to the skin of patient 12. Preferably, ultrasound sensor 58 is in direct contact with the skin adjacent to bladder 14, possibly with a thin intervening layer of the undergarment 54. Ultrasound sensor 58 produces ultrasonic waves that propagate through tissue of patient 12 and reflect off of tissue density transitions, such as bladder 14, within the patient. In this manner, ultrasound sensor 58 may detect changes in size of bladder 14 that can be correlated to voiding events and a voiding signature generated by external programmer 24.

Ultrasound sensor 58 wirelessly communicates with neurostimulator 20, but some embodiments may include a battery pack or other electronic support devices housed within undergarment 54 or worn on a belt around the waist of patient 12. The extra battery power and electronics may be necessary to power ultrasound sensor 58 for an extended period of time and reliably detect voiding signatures throughout the stimulation therapy.

While ultrasound sensor 58 is located to the right side of the abdomen of patient 12 in the example of FIG. 5, the ultrasound sensor may be located anywhere on undergarment 54. Multiple ultrasound sensors 58 may also be placed within multiple pockets 56. In alternative embodiments, undergarment 54 may house sensors other than or in addition to ultrasound sensor 58. For example, pocket 56 may hold a microphone sensor that detects sound from liquid moving within bladder 14. The sound waves are digitized into data and correlated to identify a voiding signature of the microphone sensor.

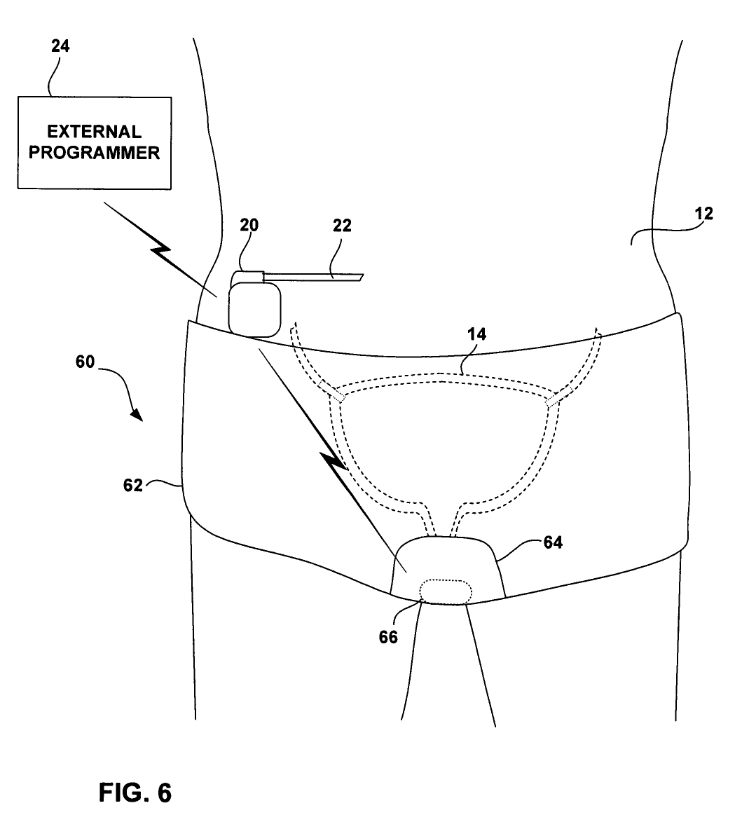

FIG. 6 is a schematic diagram illustrating an implantable stimulation system, incorporating a wearable wetting sensor that senses bladder events. As shown in FIG. 6, system 60 is similar to system 10 of FIG. 1 in that each system includes a sensor for feedback and operates similarly to learn to detect a voiding signature. System 60 includes stimulator 20, external programmer 24, and undergarment 62. Undergarment 62 includes pocket 64 that holds wetting sensor 66 near the opening of patient 12 urethra (not shown). Wetting sensor 66 transmits data to neurostimulator 20 of external programmer 24 that varies as wetting sensor 66 detects moisture. When the moisture data is compared to voiding events indicated by patient 12, external programmer 24 calculates a voiding signature that neurostimulator 20 uses to modify stimulation therapy.

Patient 12 may wear undergarment 62 under regular clothing so that wetting sensor 66 may detect voiding events or leakage events. Wetting sensor 66 detects the presence of fluid which indicates that wetting has occurred. In some cases, wetting sensor 66 may be capable of also detecting fluid pH or other characteristic of the fluid to identify that the fluid is urine. Wetting sensor 66 allows system 60 to adjust stimulation therapy according to the voiding signature detected by neurostimulator 20. In some embodiments, pocket 64 may also include absorption material that absorbs voided urine, such that undergarment 62 is similar to a diaper or protective garment. In addition, undergarment 62 may be disposable, along with wetting sensor 66.

In some embodiments, wetting sensor 66 may only transmit data to neurostimulation 20 when the wetting sensor detects wetness. Alternatively, neurostimulator 20 may only receive data intermittently or as scheduled by the clinician. These transmission reducing protocols may increase the battery life of neurostimulator 20 and wetness sensor 66. Wetness sensor 66 may sense wetness using electrical sensors that include electrodes to detect wetness based on resistive or capacitive changes. Other examples include optical moisture sensors, and chemical moisture sensors. In general, sensor 66 may sense wetness using sensor elements similar to those used for humidity and moisture testing.

In alternative embodiments, wetting sensor 66 may not be included in undergarment 62. For example, wetting sensor 66 may be included in a pad that fits in patient 12 underwear to maximize patient comfort. The pad may be gender specific, with wetting sensor 66 located near the middle for female anatomy and near the ventral side for male anatomy. In addition, wetting sensor 66 may also be located at the distal tip of a condom-like device that males may use to cover the penis.

Patient 12 may utilize a combination of multiple sensors, such as the sensors shown in any FIGS. 1-6. Multiple sensors may provide a more precise determination of the actual voiding signature, where each sensor may need to detect a certain value or characteristic before the voiding signature is identified. This determination may be completed using a classification map that defines a region of space within the classification map as a particular event. Only when each sensor provides data that overlaps over the same region of space in the classification map does neurostimulator 20 identify the voiding signature and appropriately change stimulation therapy. In some embodiments, slight stimulation changes may be made when less than all sensors indicate that a voiding event is occurring. The classification map may also allow neurostimulator 20 to modify stimulation when other types of dysfunctions are also detected.

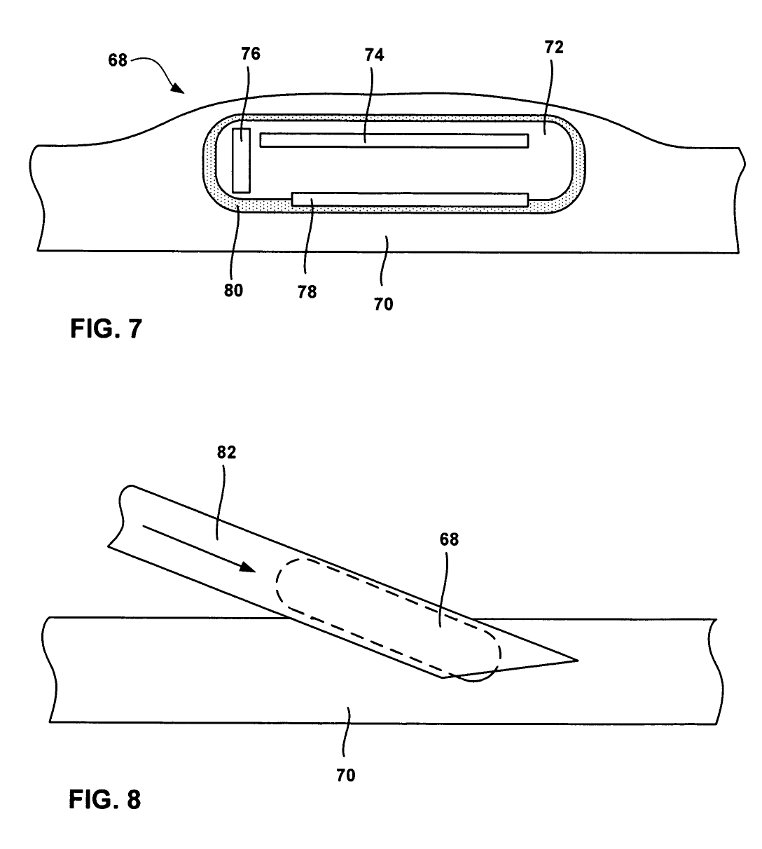

FIG. 7 is a cross-sectional side view of an implantable sensor placed within a tissue of a patient. Sensor 68 is an embodiment of sensors 26, 28 or 32 that may be implantable in the interior or exterior of bladder 14, or sensor 68 may be similar to sensors 58 and 66 that are not implanted in patient 12. Sensor housing 72 of sensor 68 is embedded in bladder wall 70 and includes circuit board 74, power source 76, and sensing element 78. Sensor housing 72 is in the shape of a rounded capsule and includes a smooth surface. Sensing element 78 extends from housing 72. In some embodiments, sensing element 78 may include a strain gauge to detect pressure, which slightly protrudes from the housing to sense deformation changes in bladder wall 70. Sensor 68 rests in wall cavity 80 formed within bladder wall 70. Sensor 68 may have a capsule-like shape, and may have a length of approximately 2 to 10 mm, a width of approximately 2 to 5 mm, and a thickness of approximately 1 to 5 mm. The capsule-like shape may produce a circular cross-section, in which case sensor 68 may have a diameter of approximately 1 to 5 mm, rather than width and height dimensions.

Sensing element 78 senses a change in deformation of bladder wall 70 as bladder 14 expands and contracts. Sensing element 78 may detect pressure changes, deflection, shear stress, electrical differentials, or other detectable parameter of bladder 14. Processing electronics on circuit board 74 detect these changes sensed by sensing element 78. Circuit board 74 communicates the bladder information to neurostimulator 20, external programmer 24, or both, e.g., by wireless telemetry. Circuit board 74 also controls the operation of sensor 68.

Implanting bladder sensor 68 within bladder wall 70 may be a simple method for securing the sensor sensing element 78. As bladder 14 expands and contracts, sensing element 78 senses the changed pressure of bladder wall 70 and indicates a change in size of the bladder or an abrupt contraction. For example, a higher force in bladder wall 70 may indicate an expanding bladder 14 or a contraction. Although sensing element 78 may be a strain gauge, many other types of sensing components may be used to sense a change in deformation of bladder 14. In the case of sensor 68 being used as a wetting sensor 38, sensing element 78 may detect the presence of a fluid.

FIG. 8 is a schematic diagram illustrating endoscopic deployment of the implantable sensor of FIG. 7. Bladder sensor 68 may be implanted through endoscopic, laparoscopic, or similar minimally invasive techniques. A surgeon makes a few small incisions in the abdomen of patient 12 and guides bladder sensor 68 within needle 82 to bladder 14 with the aid of a small camera. Needle 82 may be constructed of a metal alloy and comprise a hollow cylinder and a pointed distal end for puncturing bladder wall 70. Needle 82 includes bladder sensor 68 and a fluid to force the sensor out of the needle. An exemplary fluid may be saline or other biocompatible fluid. In other embodiments, needle 82 may comprise a catheter or other hollow delivery vehicle.

Once needle 82 in positioned at the appropriate location of bladder 14, the surgeon may force sensor 68 into place. Removing needle 82 from bladder wall 70 allows the external tissue of bladder wall 70 to close and surround sensor 68. In some embodiments, the surgeon may suture the insertion hole of bladder wall 70 to promote tissue healing. The suture may comprise resorbable or non-resorbable suture or staples. When implanting sensor 68, the inner surface of bladder wall 70 should not be breached in order to prevent patient 12 from developing infection or other health problems.

In other embodiments, bladder sensor 68 may be implanted through more invasive procedures, such as open abdominal surgery which exposes bladder 14. In some embodiments, multiple sensors 70 may be placed around bladder 14 to generate an average expansion or contraction of the entire bladder.

Bladder sensor 68 has a biocompatible housing, which may be formed from titanium, stainless steel or other materials. In some embodiments, bladder sensor 68 may carry one or more expandable elements that help to anchor the sensor within the bladder wall. The expandable elements may be constructed from a hydrogel material. During implantation, the expandable elements are in a dehydrated state, in which the expandable elements are smaller. But when implanted in the body of a patient, the expandable elements absorb water from the body tissues and assume a hydrated state. In the hydrated state, the expandable elements have a larger perimeter. Expansion of the expandable elements resists migration of the sensor 68 within bladder wall 70.

FIG. 9 is a cross-sectional side view of an implantable sensor attached to a tissue of a patient. Sensor 84 may be an embodiment of sensors 26, 28, 32 and 38, which is attachable within patient 12. As shown in FIG. 9, sensor 84 includes a sensor housing 88 and sensing element 94 that extends from the housing. Sensing element 94 may be a strain gauge sensor that senses mechanical deformation of the wall of bladder 14. Sensing element 94 may be coupled to a circuit board 90 within sensor 84. A power source 92, such as a battery, may be provided to power circuit board 90, sensing element 94 or both. Circuit board 90 includes processing electronics to process signals generated by sensing element 94, and generate bladder information based on the signals. In addition, circuit board 90 may include telemetry circuitry for wireless telemetry with neurostimulator 20, external programmer 24, or both. Sensor 84 is attached to bladder wall 86 by fastening pin 100 through tissue 87. Vacuum channel 96 applies negative pressure in vacuum cavity 98 to draw in a portion of bladder wall 86, i.e., tissue 87.

Power source 92 may take the form of a small rechargeable or non-rechargeable battery, which may be configured as a coin cell or pin cell. Different types of batteries or different battery sizes may be used, depending on the requirements of a given application. To promote longevity, power source 92 may be rechargeable via induction or ultrasonic energy transmission, and includes an appropriate circuit for recovering transcutaneously received energy. For example, power source 92 may include a secondary coil and a rectifier circuit for inductive energy transfer. Power generation or charging electronics may be carried on circuit board 90. In still other embodiments, power source 92 may not include any storage element, and sensor 84 may be fully powered via transcutaneous inductive energy transfer. As a further alternative, neurostimulator 20 or programmer 24 may be configured to apply inductive power to sensor 84 whenever sensing is desired. In this case, when inductive power is not applied, sensor 84 is asleep. Upon application of inductive power, sensor 84 wakes up, acquires a sense signal, and transmits the signal to programmer 24 or neurostimulator 20. Accordingly, neurostimulator 20 or programmer 24 determines the sampling rate of sensor 84 by powering up the sensor at desired intervals.

In the exemplary embodiment of FIG. 9, sensor 84 includes a strain gauge as sensing element 94 to sense mechanical deformation of the wall of bladder 14 and thereby indicate changes in bladder 14 size or shape or sense contractions. Sensing element 94 senses the stretch of bladder 14 to detect the expansion and contraction, or increase and decrease, in size of bladder 14, and thereby senses if voiding has occurred. The expansion and contraction may be monitored as gradual or instantaneous changes. For example, gradual expansion may indicate a gradual filling of bladder 14, while a rapid or instantaneous change may indicate a bladder muscle contraction and the possibility of imminent, involuntary voiding.

The disclosure is not limited to the use of a strain gauge for sensing or detecting changes in the size, wall thickness, shape or volume of bladder 14. For example, other embodiments may include one or more electrodes for sensing the electrical activity of the muscles surrounding bladder 14. Detecting muscle activity may be correlated with changes in bladder size or contraction. In other embodiments, sensor 84 may utilize an ultrasound transducer to sense the thickness of the wall of bladder 14 or the distance to the opposite wall of bladder 14. Further, sensor 84 may contain more than one sensing component, such as two strain gauges. In each case, sensor 84 is deployed on or within an exterior wall of bladder 14.

Strain gauge sensing element 94 may be formed with a flexible material, including polyurethane or silicone. In other embodiments, the strain gauge may be formed with a flexible polymer or metal alloy. The strain gauge may be able to sense small changes in bladder 14 wall stretch or deformation for detection of voiding events and the voiding signature. The strain gauge may carry a circuit containing resistive elements, which may be printed, deposited or otherwise formed on the flexible material. In some embodiments, the strain gauge may include small protrusions or adhesion points with stick to certain locations on bladder wall 86. As bladder wall 86 expands or contracts, these locations will move with respect to each other.

Strain gauge sensing element 94 senses the movement of bladder wall 86 in terms of changes in impedance, voltage, or other electrical characteristics of the circuit formed on the strain gauge to sense the expansion or contraction of bladder 14. Processing electronics carried by circuit board 90, or carried by neurostimulator 20 or external programmer, process the sensed bladder condition or activity signal to detect expansion or contraction of the bladder 14. In particular, the signal output by sensing element 94 can be used to sense a urine fill stage of bladder 14, which may be indicative of progression toward a voiding event, or a muscle contraction, which may be indicative of the voiding signature.

The electrical characteristics may be monitored for rapid or instantaneous changes indicative of bladder contraction, as well as slow, gradual changes indicative of bladder filling. Rapid and gradual changes may both indicate progression of the bladder toward an imminent voiding event. For example, contraction may result in an immediate leakage of urine, while bladder filling may result in an eventual leakage of urine when the bladder becomes too full. In both cases, the events are logged to provide feedback to neurostimulator 20. The characteristics measured by sensing element 94 and processing electronics carried by circuit board 90 may be sent to neurostimulator 20 or programmer 24 as raw measurements or as bladder condition or activity signals indicating a bladder condition, such as a voiding state.

Sensor housing 88 may be made from a biocompatible material such as titanium, stainless steel or nitinol, or a polymeric material such as silicone or polyurethane. Another material for fabrication of sensor housing 88 is a two-part epoxy. An example of a suitable epoxy is a two-part medical implant epoxy manufactured by Epoxy Technology, Inc., mixed in a ratio of 10 grams of resin to one gram of activator. In general, sensor housing 88 contains no external openings, with the exception of the opening containing sensing element 94, thereby protecting power source 92 and circuit board 90 from the environment within bladder 14. The opening in sensor housing 88 that receives sensing element 94 is sealed to prevent exposure of interior components.

In some embodiments, sensor housing 88 may have a capsule-like shape with a length in a range of approximately 2 to 15 mm, a width in a range of approximately 2 to 10 mm, and a height in a range of approximately 2 to 10 mm. The capsule-like shape may produce a circular cross-section, in which case sensor housing 88 may have a diameter of approximately 3 to 10 mm, rather than width and height dimensions. Vacuum cavity 98 may be sized to capture a volume of bladder wall tissue on the order of approximately 1 to 5 mm³.

Inward deflection of sensing element 94 may signal the expansion of bladder 14. This expansion may be due to the gradual addition of urine in the bladder or a contraction of muscle in bladder wall 86. During expansion of bladder 14, neurostimulator 20 may provide electrical stimulation to enhance pelvic floor tone or urinary sphincter function, for example, to keep urine within the bladder. Once sensing element 94 indicates a sufficiently large expansion, electronics on circuit board 90 generate bladder information based on the expansion. Sensor 84 may communicate the information directly to external programmer 24 or neurostimulator 20 by wireless telemetry. In other embodiments, sensor 84 may be coupled to implantable neurostimulator 20 by a wired connection.

Sensor 84 may transmit bladder information substantially continuously or periodically, e.g., every few seconds or minutes. In some embodiments, sensor 84 may transmit bladder information when there is an abrupt change sensed by sensing element 94, e.g., a deformational change that exceeds a predetermined threshold, indicating a contraction.

Attaching implantable sensor 84 to the bladder wall 86 of bladder 14 may be accomplished in a variety of ways, but preferably is completed in a manner that will not excessively injure bladder 14 or otherwise cause excessive trauma during implantation. Preferably, attachment should cause limited inflammation and substantially no adverse physiological modification, such as tissue infection or a loss in structural integrity of bladder 14. However, it is desirable that implantable sensor 84 also be attached securely to the attachment site in order to provide an extended period of measurement without prematurely loosening or detaching from the intended location.

As an example, sensor housing 88 may contain a vacuum cavity 98 that permits a vacuum to be drawn by a vacuum channel 96. The vacuum is created by a deployment device having a vacuum line in communication with vacuum channel 96. The vacuum draws tissue 87 from bladder wall 86 into vacuum cavity 98. Once tissue 87 of bladder wall 86 is captured within vacuum cavity 98, a fastening pin 100 is driven into the captured tissue to attach sensor housing 88 within bladder 14. Fastening pin 100 may be made from, for example, stainless steel, titanium, nitinol, or a high density polymer.

The shaft of pin 36 may be smooth or rough, and the tip may have a sharp point to allow for easy penetration into tissue. Fastening pin 100 may be driven into housing 88 and tissue 87 of bladder wall 86 under pressure, or upon actuation by a push rod, administered by a deployment device. In another embodiment, implantable sensor 84 may be attached without the use of a penetrating rod but with a spring-loaded clip to pinch trapped bladder wall 86 within cavity 98. A variety of other attachment mechanisms, such as pins, clips, barbs, sutures, helical screws, surgical adhesives, and the like may be used to attach sensor housing 88 to bladder wall 86 of bladder 14.

In the example of FIG. 9, sensor housing 88 of implantable sensor 84 is attached to the interior wall of bladder 14. However, the attachment site for sensor housing 88 could be at any position on bladder wall 86 that does not interfere with bladder function or other organ function. For example, sensor housing 88 may be placed in the top of the bladder or near the urethra. In some patients, the most desirable position may coincide with the least invasive implantation surgery. Sensor 84 may be surgically implanted using open surgery or laparoscopic techniques.

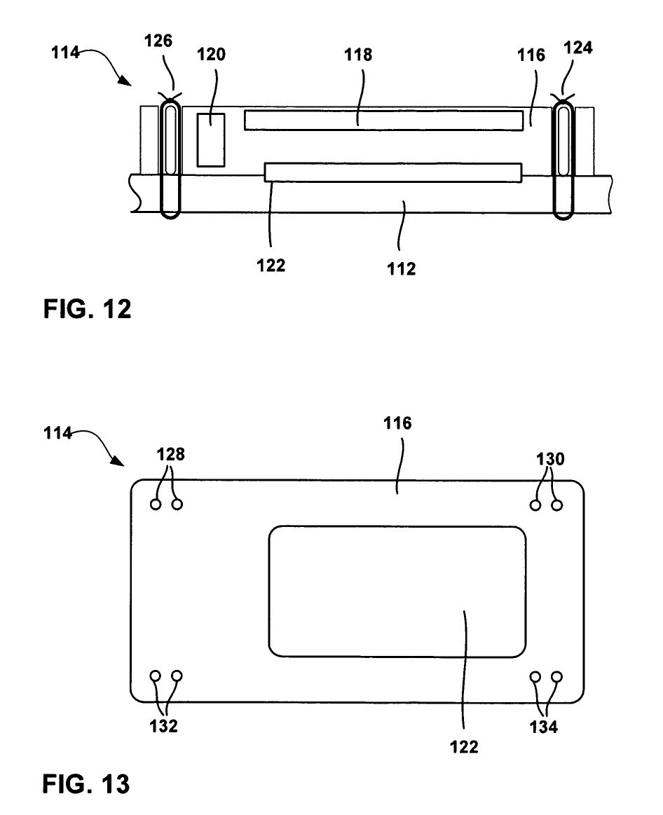

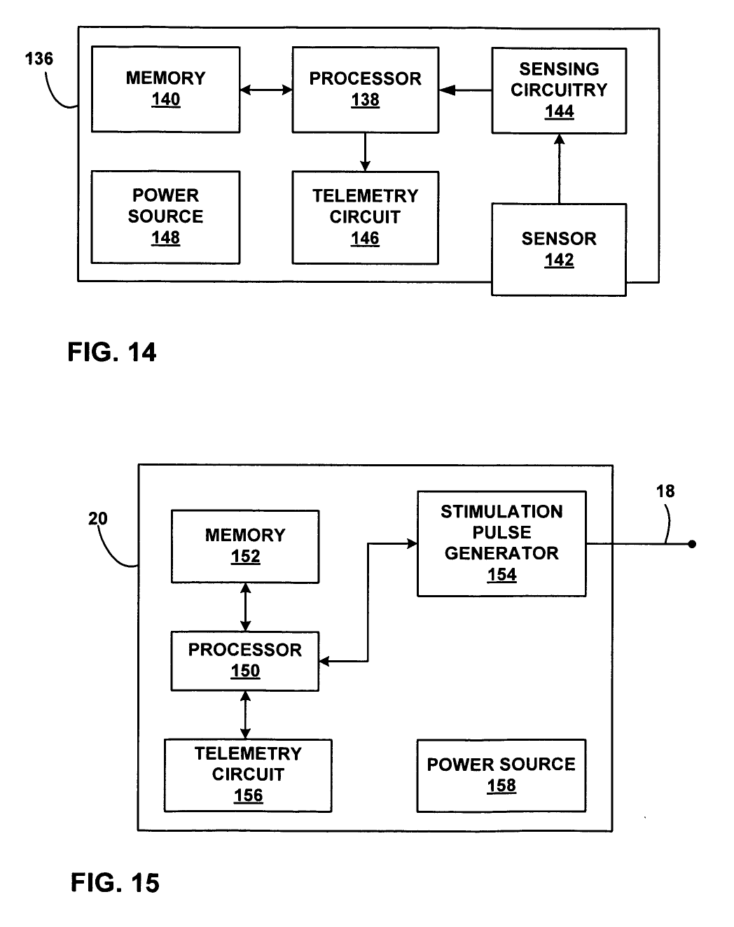

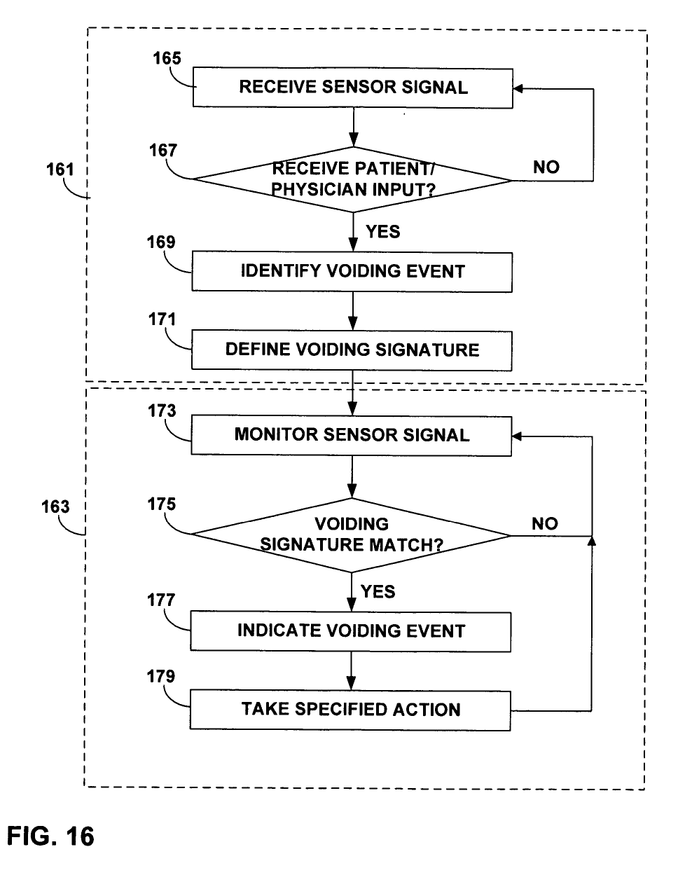

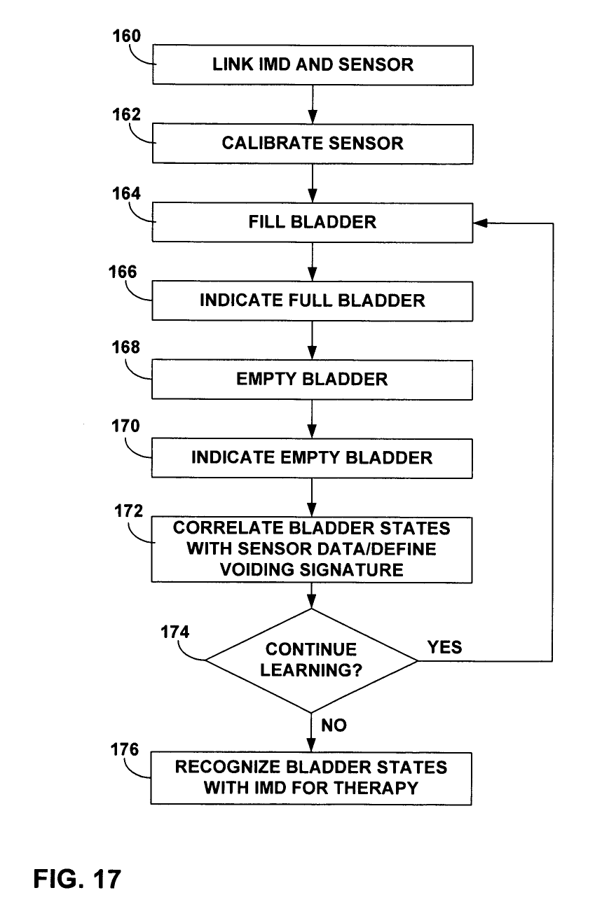

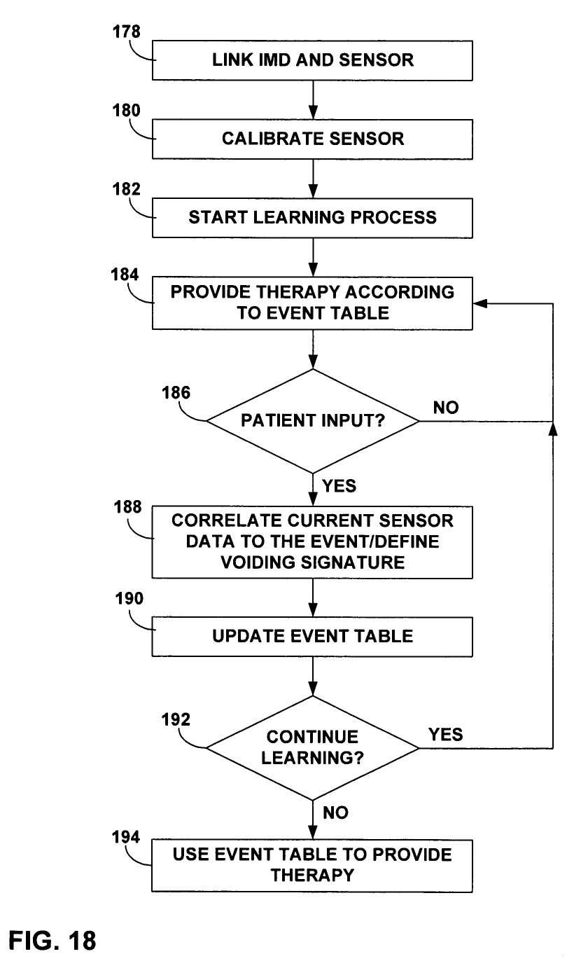

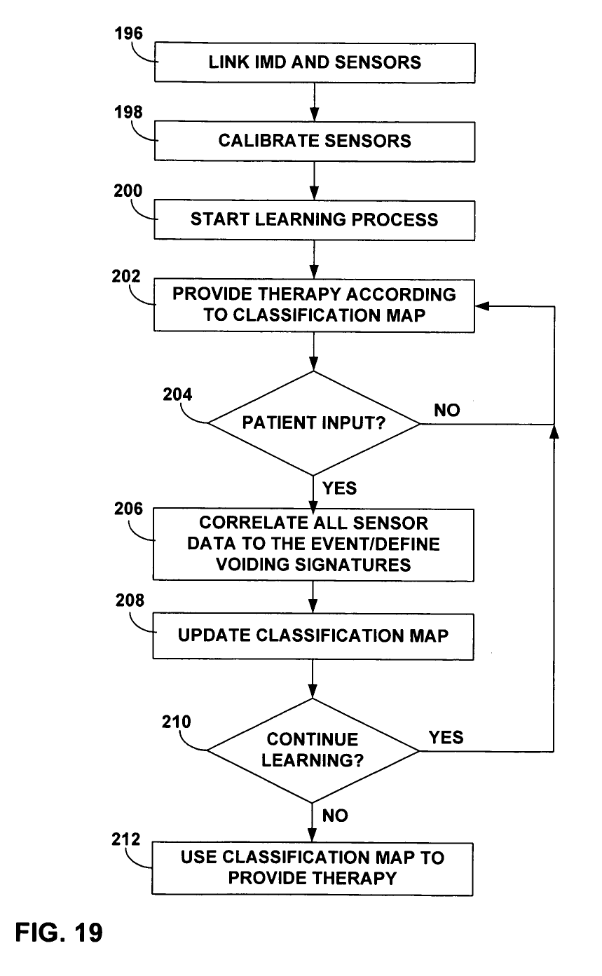

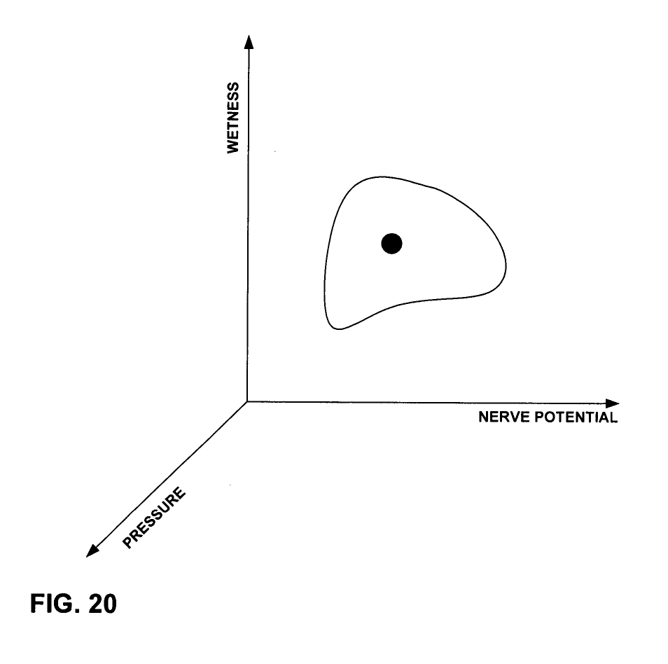

FIG. 10 is a bottom view of the implantable sensor of FIG. 9. Sensor housing 88 includes sensing element 94 and vacuum cavity 98, which come into contact with bladder wall 86. While sensing element 94 is rectangular and large with respect to sensor housing 88 to contact a large surface area of bladder wall 86, some embodiments may include two or more sensing elements, such as strain gauges of similar or different shapes. For example, housing 88 may include a sensing element on each end of housing 88 separated by vacuum cavity 98.

Vacuum cavity 98 holds a portion of tissue from bladder wall 86 in order to keep sensing element 94 in contact with the exterior surface of bladder 14. In some embodiments, sensor housing 88 may contain more than one vacuum cavity to attach to multiple points along bladder wall 86. For example, one vacuum cavity on each end of housing 88 may provide secure contact between sensing element 94 and bladder wall 86. In other embodiments, housing 88 may be formed into a different shape than a rectangle. For example, housing 88 may comprise a circular shape or concave shape to better fit the curvature of bladder 14.

FIG. 11 is a cross-sectional side view of a deployment device during deployment and fixation of the implantable sensor of FIG. 9. In the example of FIG. 11, deployment device 102 includes a distal head 104. Distal head 104 may be mounted on an elongated sheath 106 (partially shown in FIG. 11) configured for laparoscopic introduction into patient 12 through a trocar. Deployment device 102 may be used with other laparoscopic components, such as a gas distension tube for inflating the pelvic cavity to facilitate access to bladder 14, and a visualization scope for viewing the implantation site. In some embodiments, visualization components may be integrated with deployment device 102.

As shown in FIG. 11, distal head 104 receives a vacuum line 108 and a positive pressure line 110 via elongated sheath 106. Vacuum line 108 is coupled to a vacuum outside of patient 12 via a tube or lumen extending along the length of deployment device 102. Similarly, positive pressure line 110 is coupled to a positive pressure source (not shown) via a tube or lumen extending along the length of deployment device 102. Vacuum line 108 is in fluid communication with vacuum channel 96 and vacuum cavity 98, and permits the physician to draw a vacuum and thereby capture tissue 87 of bladder wall 86 within the vacuum cavity. Positive pressure line 110 permits the physician to apply a pulse of high pressure fluid, such as a liquid or a gas, to drive fixation pin 100 into sensor housing 88 and through tissue 87 of bladder wall 86. Pin 100 thereby fixes sensor housing 88 to external bladder wall 86. In some embodiments, a membrane mounted over an opening of positive pressure line 110 may be punctured by pin 100.

Once fixation pin 100 attaches sensor 84 to bladder 14, vacuum line 108 is no longer needed. However, in some embodiments, vacuum line 108 may be used to detach pressure sensor 84 from distal head 104 of deployment device 102. By terminating vacuum pressure, or briefly applying positive pressure through vacuum line 108, for example, head 104 may separate from sensor 84 due to the force of the air pressure. In this manner, vacuum line 108 may aid in detachment of sensor 84 prior to removal of deployment device 102.

As described previously in FIG. 9, fixation pin 100 punctures bladder wall 86 for fixation of sensor 84. While the force of this fixation may vary with patient 12, deployment device 102 provides adequate force for delivery of pin 100. In an exemplary embodiment, positive pressure line 110 is completely sealed and filled with a biocompatible fluid (such as water, saline solution or air). Sealing the end of positive pressure line 110 is fixation pin 100 or a head on fixation pin 100.

Fixation pin 100 is generally able to move within positive pressure line 110 much like a piston. Force to push fixation pin 100 through tissue 87 of bladder wall 86 captured in vacuum cavity 98 is created by application of a pulse of increased fluid pressure within positive pressure line 110. For example, the physician may control a positive pressure source via control handle attached to deployment device 102. This simple delivery method may provide high levels of force, allow multiple curves and bends in deployment device 102, and enable a positive pressure line 110 of many shapes and sizes.

In an alternative embodiment, a flexible, but generally incompressible, wire may be placed within positive pressure line 110 and used as a push rod to force fixation pin 100 through the captured tissue 87 of bladder wall 86. This wire presents compressive force from the control handle of deployment device 102 directly to fixation pin 100. This method may eliminate any safety risk of pressurized fluids entering patient 12 or, in some embodiments, permit retraction of pin 100 after an unsuccessful fixation attempt. If attached, the flexible wire may be attached to pin 100 and pulled back to remove the pin from tissue 87. The flexible wire may be sheared from fixation pin 100 for detachment purposes as distal head 104 releases sensor 84. This detachment may be facilitated by a shearing element or low shear stress of the wire.

In FIG. 11, deployment device 102 illustrates the attachment of vacuum line 108 and positive pressure line 110 to one end of sensor 84. In some embodiments, deployment device 102 may attach vacuum line 108 and positive pressure line 110 to their respective channels opening on the top of sensor housing 88 instead of the side of sensor housing 88. This change in location may facilitate attachment of sensor 84 from a variety of locations or on certain locations on the outside of bladder 14.

Deployment device 102 is introduced to patient 12 by a small incision in the abdomen of the patient. A surgeon may guide distal head 104 through the abdominal space to the exterior of bladder 14. Once at bladder 14, the surgeon locates the desired spot for attaching sensor 84. Sensor 84 is then pressed up against bladder wall 86 and the vacuum is initiated to bring tissue 87 into vacuum cavity 98 before fixation pin 100 is driven through tissue 87. Deployment device releases sensor 84 and is removed from patient 12.

In other embodiments, sensor 84 may be attached to bladder 14 through open abdominal surgery to precisely locate the attachment point on bladder 14. In this type of procedure, deployment device 102 may or may not be used to attach sensor 84 to bladder wall 86. In some embodiments, deployment device 102 may include a small endoscopic camera in the distal head 104. The camera may enable the physician to better guide deployment device 102 through a small opening in patient 12 to a desired attachment location on the external surface of bladder 14 in less time with more accuracy, as is common in endoscopic surgery. Images may be displayed using video fed to a display monitor.

Distal head 104 may be disposable. Disposable devices that come into contact with patient 12 tissues and fluids greatly decrease the possibility of infection in implantable devices. In other embodiments, the entire deployment device 102 may be manufactured from robust materials intended for multiple uses. The device would then need to be sterilizable between uses. In still a further embodiment, the features of distal head 104 may be incorporated into sensor 84. In this configuration, sensor 84 may be larger in size but would include the necessary elements for attachment within the device. After attachment, the entire sensor would detach from the handle of deployment device 102, reducing the difficulty of removing the entire deployment device 102, including distal head 104.

After the useful life of implantable sensor 84 is complete or it is no longer needed within patient 12, it can be removed from patient 12 in some manner. Alternatively, sensor 84 may simply remain in place. As an example, deployment device 102 may be reinserted into patient 12, navigated to bladder 14, and reattached to sensor 84. Deployment device 102 may then be withdrawn from bladder 14, explanting sensor 84 from patient 18. Alternatively, a surgeon may perform open abdominal surgery to remove the implanted sensor 84 and neurostimulator 20.

FIG. 12 is an enlarged schematic diagram illustrating an implantable sensor sutured to a tissue of a patient. As shown in FIG. 12, sensor 114 is an embodiment of sensors 26, 28, 32, 38, 58 and 66. In the case of sensors 26, 28, 32, and 38, sensor 114 is sutured to bladder 14. In the case of sensors 58 and 66, sensor 114 may be sewn to a pocket of an undergarment. Sensor housing 116 is attached to bladder wall 112 and includes circuit board 118, power source 120, and sensing element 56. Sutures 124 and 126 are used to attach bladder sensor 114 to bladder wall 112. Although only two sets of sutures can be shown in FIG. 12, sensor 114 may include four or more sets, one at each corner of the rectangular shaped sensor.

Circuit board 118, power source 120 and sensing element 56 may all be similar to circuit board 90, power source 92 and strain gauge 94 of FIG. 9. In addition, sensor housing 116 may be functionally similar to sensor housing 88 of FIG. 9. Differences between these components of each embodiment may relate to only the size or shape of each component. As in some embodiments of sensing element 94, sensing element 122 may include a strain gauge sensor that senses a change in deformation of bladder wall 112 as bladder 14 expands and contracts. Sensing element 122 sends the bladder information to circuit board 118. Circuit board 118 wirelessly communicates the bladder information to neurostimulator 20, external programmer 24, or both. Circuit board 118 also may control the operation of sensor 114.

Bladder sensor 114 may be implanted through laparoscopic techniques. For example, a surgeon may make a few small incisions in the abdomen of patient 12 and guide bladder sensor 114 to bladder 14 with the aid of a small camera. Once sensor 114 is placed on the external surface bladder wall 112, the surgeon uses sutures to tie sensor 114 to bladder wall 112, which is illustrated by sutures 124 and 126 in FIG. 12. The sutures may or may not penetrate through bladder wall 112, and no urine will escape bladder 14 in either case.

In other embodiments, bladder sensor 114 may be implanted through more invasive procedures, such as open abdominal surgery which exposes bladder 14. In some embodiments, metal or plastic staples may be used to fix sensor 16 to bladder wall 112 instead of nylon sutures. In some embodiments, multiple sensors 114 may be placed around bladder 14 to generate an average expansion or contraction of the entire bladder.

Once attached to bladder wall 112, sensing element 122 may be securely forced against bladder wall 112. As bladder 14 expands and contracts, sensing element 122 may sense the changed pressure by bladder wall 112 and indicate a change in size of the bladder. Similar to sensing element 94 of FIG. 9, many other types of sensing components may be used to sense a change in deformation of bladder 14. However, a strain gauge is described herein for purposes of illustration.

FIG. 13 is an enlarged, bottom view of the implantable sensor of FIG. 12. Bladder sensor 114 includes sensor housing 116 and sensing element 122. Fixation holes 128, 130, 132 and 134 are voids in housing 116 and allow suture to be passed through housing 116 in order for sensor 114 to be attached to bladder wall 112. Sensing element 122 may occupy a majority of the surface area of bladder sensor 114 that contacts bladder wall 112. While sensing element 122 is rectangular in shape, the strain gauge may be formed of any symmetric or asymmetrical shape. In the example of FIGS. 12 and 13, sensor 114 may have a patch-like shape, and may have a length of approximately 2 to 15 mm, a width of approximately 2 to 10 mm, and a thickness of approximately 2 to 10 mm.

Fixation holes 128, 130, 132 and 134 each contain a pair of passages through housing 116. Each pair of passages is located near a corner of housing 116. A surgeon may pass a suture through these holes to attach housing 116 to bladder 14 in a desired location of bladder wall 112. While fixation holes 128, 130, 132 and 134 each contain two holes, other embodiments may include more or less holes in housing 116. For example, each corner of housing 116 may only contain one hole. Suture would then pass through the hole and around the outside of housing 116. As a further example, each corner may contain three holes for further securing housing 116 to bladder wall 112.

Other fixation methods to secure bladder sensor 114 to bladder wall 112 may include other structures different than sutures. For example, each corner of housing 116 may contain a barbed needle or helical screw that ejects from housing 116 into bladder wall 112. The barbed needles may secure sensor 114 to bladder 14 without lengthy attachment procedures. Also, surgical adhesives may be used as an alternative, or in addition to, mechanical fasteners such as sutures, needles or screws.

FIG. 14 is a functional block diagram illustrating various components of an exemplary implantable sensor. As shown in FIG. 14, bladder sensors 26, 28, 32, 38, 58 and 66, described herein as sensor 136. In the example of FIG. 14, sensor 136 includes a processor 138, memory 140, sensing circuitry 144, telemetry circuit 146, power source 148 and sensor 142. Sensing circuitry 144 may be carried on a circuit board, along with processor 138, memory 140 and telemetry circuit 146. Sensor 142 may be any sensor such as a pressure sensor, impedance sensor, ultrasound sensor, wetness sensor, pH sensor, or any other sensor that transforms mechanical, chemical or electrical conditions into electrical signals representative of physiological function of bladder 14. The electrical signals may be amplified, filtered, and otherwise processed as appropriate by sensing circuitry 144 within sensor 136. In some embodiments, the signals may be converted to digital values and processed by processor 138 before being saved to memory 140 or sent to neurostimulator 20 via telemetry circuit 146.

Memory 140 stores instructions for execution by processor 138 and bladder information generated by sensing circuitry 144. Bladder data may then be sent to neurostimulator 20 or external programmer 24 for long-term storage and retrieval by a user. Memory 140 may include separate memories for storing instructions and bladder information. In addition, processor 138 and memory 140 may implement loop recorder functionality in which processor 138 overwrites the oldest contents within the memory with new data as storage limits are met, thereby conserving data storage resources within sensor 136. Alternatively, sensor 136 may be configured to immediately transmit sensed information to another device such as neurostimulator 20 or external programmer 24, in which case memory, processing overhead, and power consumption in sensor 136 can be substantially reduced.

Processor 138 controls telemetry circuit 146 to send bladder information to neurostimulator 20 or external programmer 24 on a continuous basis, at periodic intervals, or upon request from the implantable stimulator or programmer. The bladder information may be a pre-processed indication of a voiding event, in the case that sensor 136 includes the processing intelligence to analyze the sensed signals for a voiding signature. Alternatively, the bladder information may be raw sensor data obtained by sensor 136. In this case, neurostimulation 20 or external programmer 24 may provide the processing intelligence to analyze the sensed signals for a voiding signature. Wireless telemetry may be accomplished by radio frequency (RF) communication or proximal inductive interaction of sensor 136 with external programmer 24.

Power source 148 delivers operating power to the components of sensor 136. Power source 148 may include a small rechargeable or non-rechargeable battery and a power generation circuit to produce the operating power. Recharging may be accomplished through proximal inductive interaction between an external charger and an inductive charging coil within sensor 136. In some embodiments, power requirements may be small enough to allow sensor 136 to utilize patient motion and implement a kinetic energy-scavenging device to trickle charge a rechargeable battery. In other embodiments, traditional batteries may be used for a limited period of time. As a further alternative, an external inductive power supply could transcutaneously power sensor 136 whenever measurements are needed or desired.

FIG. 15 is a functional block diagram illustrating various components of an implantable stimulator that communicates wirelessly with an implantable sensor. In the example of FIG. 15, neurostimulator 20 includes a processor 150, memory 152, stimulation pulse generator 154, telemetry circuit 156, and power source 158. Memory 152 may store instructions for execution by processor 150, stimulation therapy data, and bladder information received from sensors 26, 28, 32, 38, 58 or 66 via telemetry interface. Bladder information is received and may be recorded for long-term storage and retrieval by a user, and adjustment of the stimulation parameters. Memory 152 may include separate memories for storing instructions, bladder information, and voiding signature information.

Processor 150 controls stimulation pulse generator 154 to deliver electrical stimulation therapy via one or more leads 22. Processor 150 controls telemetry circuit 156 to send and receive information. An exemplary range of neurostimulation stimulation pulse parameters likely to be effective in treating incontinence, e.g., when applied to the sacral or pudendal nerves, are as follows:

1. Frequency: between approximately 0.5 Hz and 500 Hz, more preferably between approximately 5 Hz and 250 Hz, and still more preferably between approximately 10 Hz and 50 Hz.

2. Amplitude: between approximately 0.1 volts and 50 volts, more preferably between approximately 0.5 volts and 20 volts, and still more preferably between approximately 1 volt and 10 volts. The amplitude may be representative of a biological load between 10 ohms and 10,000 ohms.

3. Pulse Width: between about 10 microseconds and 5000 microseconds, more preferably between approximately 100 microseconds and 1000 microseconds, and still more preferably between approximately 180 microseconds and 450 microseconds.

Based on bladder information received from one or more sensors, processor 150 interprets the information and determines whether stimulation parameters should be changed. For example, processor 150 may perform statistical analyses of the detected bladder information to determine if the efficacy of therapy necessitates a change in the parameters. Information may be received from sensors 26, 28, 32, 38, 58 or 66 on a continuous basis, at periodic intervals, or upon request from neurostimulator 20 or external programmer 24. Alternatively, or additionally, sensors 26, 28, 32, 38, 58 or 66 may transmit bladder information when there is an abrupt change in the physiological function of bladder 14, e.g., indicating contraction at the onset of involuntary leakage.

Processor 150 may correlate the bladder information with feedback from patient 12 indicating voiding events to determine the voiding signature and store data representing the voiding signature in memory 152. Alternatively, memory 152 may only store the voiding signature generated by external programmer 24. Stimulation pulse generator 154 provides electrical stimulation according to the stored stimulation parameters, which may change if the voiding signature is detected.

Bladder function or mechanical properties may change due to a variety of factors, such as an activity type, activity level or posture of the patient 12. Hence, for a given set of stimulation parameters, the efficacy of stimulation may vary in terms of rate of bladder expansion or contraction, due to changes in the physiological condition of the patient. For this reason, the continuous or periodic availability of bladder information from implantable sensors 26, 28, 32, 38, 58 or 66 is highly desirable.

With this bladder information, i.e., bladder data, neurostimulator 20 is able to monitor therapy efficacy and change the therapy when a voiding signature is detected. In particular, the new stimulation may be capable of improving pelvic floor tone or causing constriction of the urinary sphincter and thereby avoid involuntary leakage. In some cases, the adjustment may be nearly instantaneous, yet prevent leakage.

As in the case of sensors 26, 28, 32, 38, 58 or 66, wireless telemetry in neurostimulator 20 may be accomplished by radio frequency (RF) communication or proximal inductive interaction of neurostimulator 20 with sensors 26, 28, 32, 38, 58 or 66 or external programmer 24. Accordingly, telemetry circuit 156 may be similar to telemetry circuit 156. Also, power source 158 of neurostimulator 20 may be constructed somewhat similarly to power source 148 of FIG. 14. For example, power source 158 may be a rechargeable or non-rechargeable battery, or alternatively take the form of a transcutaneous inductive power interface.

FIG. 16 is a flow chart illustrating a technique for teaching an implantable medical device to identify bladder voiding events. More particularly, FIG. 16 shows a learning mode 161 and an operational mode 163. In the learning mode, a sensor signal is received from an implantable sensor (165) as described herein. The sensor signal may be received by an implantable neurostimulator or an external device such as a patient programmer or physician programmer. As the sensor signal is monitored, if the patient provides input indicating a natural voiding event, or the physician actively fills the bladder via a catheter to induce or simulate an actual voiding event (167), the neurostimulator or external device identifies a voiding event (169).

Using the received sensor signal, upon indication of a voiding event (169), the neurostimulator or external device defines a voiding signature. The voiding signature may include one or more characteristics of the sensor signal, such as amplitude, frequency, time intervals, morphology, or the like. Also, in some embodiments, the sensor signal associated with the voiding event may be stored as a voiding signature template for correlation with subsequently received signals to identify voiding events. If there is no received patient or physician input, the process continues to monitor the received sensor signal and monitor for received patient or physician input in order to define the voiding signature. Upon defining the voiding signature, it may be used in an operational mode 163 by an implantable neurostimulator and/or external device.

For example, the neurostimulator or external device monitors the sensor signal received from the sensor (173), and compares it to the voiding signature to determine whether there is a substantial voiding signature match (175). If not, the sensor signal continues to be monitored. If a voiding signature match is detected (175), however, the neurostimulator and/or external device indicates a voiding event (177) and takes specified action (179). The specified action may include modification of stimulation to permit or prevent the voiding event, and/or recording of the indicated voiding event in a log. Although the monitoring of the sensor signal for a voiding signature match is described above as being performed by a neurostimulator or external device, in some embodiments, the sensor itself may be equipped to process the sensor signal and compare it to the voiding signature.

FIG. 17 is a flow chart illustrating a technique for teaching an implantable medical device to identify bladder events from a sensor in a clinic before therapy begins. In the example of FIG. 17, a clinician provides input by inducing a simulated voiding event. In particular, the clinician fills the bladder with fluid via a catheter to cause the patient to void the bladder contents. As shown in FIG. 17, the clinician links neurostimulator 20, i.e. the implantable medical device (IMD), and the sensor that will be used to detect the voiding signature (160). The clinician calibrates the sensor based upon the precise location of the sensor (162), and the clinician begins to fill bladder 14 of patient 12 (164). Once bladder 14 is full, the clinician uses external programmer 24 to indicate that the bladder is full (166)

Once full, the clinician waits for patient 12 to empty bladder 14 (168), and the clinician indicates that the bladder is empty using programmer 24 (170). External programmer 24 correlates the indicated bladder states with sensor data generated at the same time as the bladder states (172). This correlation provides the voiding signature that neurostimulator 20 will recognize to modify therapy.

If the clinician desires external programmer 24 to continue learning (174), the clinician again fills bladder 14 (164). If no more learning is desired (174), external programmer 24 transmits the voiding signature to neurostimulator 20 to recognize changes in patient urodynamic function (176). The voiding signature may continue to be updated by neurostimulator 20 or anytime that external programmer 24 conriects to the sensor and the neurostimulator.

FIG. 18 is a flow chart illustrating a technique for teaching an implantable medical device to identify bladder events from a sensor during patient therapy. In the example of FIG. 18, a patient provides input concerning actual voiding events. As shown in FIG. 18, neurostimulator 20 links to the sensor (178), and the sensor is calibrated (180). External programmer 24 begins the learning process by acquiring data from the sensor (182). Stimulation therapy is delivered to patient 12 according to the event table that includes the voiding signature (184). If the patient does not provide input to external programmer 24 (186), stimulation therapy continues without changes (184). In some embodiments, external programmer 24 may request input from patient 12 if no input is received.

If patient 12 provides input identifying a natural voiding event to external programmer 24 (186), the programmer correlates the current sensor data to the input event (188). External programmer 24 then updates the event table according to the correlated data (190). This updated event table may include a new voiding signature.

If external stimulator 24 is directed to continue learning (192), therapy is continued to be delivered to patient 12 (184). If the learning mode is no longer continued (192), neurostimulator 20 stores the event table and provides stimulation therapy according to the event table and the voiding signature (194). In some embodiments, neurostimulator 20 always learns from the data of the sensor to optimize the stimulation therapy for patient 12. Alternative embodiments may include programmer 24 learning from the data of the sensor to generate the voiding signature.

In other embodiments, patient 12 input may not be required for a voiding signature to be generated. Programmer 24 or neurostimulator 20 may use other implanted sensors preset to detect any voiding events. In this manner, the voiding signature may be generated through correlation of one or more preset sensors with the sensor that is to detect a voiding signature after the learning mode is completed.

FIG. 19 is a flow chart illustrating a technique for teaching an implantable medical device to identify bladder events from multiple sensors during patient therapy. In the example of FIG. 19, the patient provides input concerning actual voiding events. As shown in FIG. 19, neurostimulator 20 links to the sensor (196), and the sensors are calibrated (198). External programmer 24 begins the learning process by acquiring data from the sensors (200). Stimulation therapy is delivered to patient 12 according to the classification map that defines the regions for each sensor involved in the stimulation system (202). The classification map essentially defines the voiding signature when one or more sensors indicate that voiding is likely to be occurring. If the patient does not provide input to external programmer 24 (204), stimulation therapy continues without changes (202).

If patient 12 provides input to external programmer 24 (204), the programmer correlates all sensor data to the event indicated by patient 12 (206). External programmer 24 then updates the classification map according to the correlated data (208). This updated classification may define a new region that is indicative of a new voiding signature.

If external stimulator 24 is directed to continue learning (210), therapy is continued to be delivered to patient 12 (202). If the learning mode is no longer continued (210), neurostimulator 20 stores the classification map and provides stimulation therapy according to the map and the voiding signature of the map (212). In some embodiments, neurostimulator 20 always learn from the data of the sensor to optimize the stimulation therapy for patient 12.

In general, a voiding signature may refer to one or more characteristics of a sensed signal that have been correlated with a voiding event or a particular stage of a voiding event, e.g., onset, middle, or end. If multiple sensors are used, each sensor may have its own voiding signature. A cross-correlation of voiding signatures from multiple sensors may be used to identify a voiding event with greater confidence. The characteristics of a sensed signal for purposes of a voiding signature may vary greatly according to the type of sensor. In some sensors, the voiding signature may simply be a threshold crossing. In a pressure sensor, for example, a signal excursion that exceeds a predetermined threshold may be used to detect a pressure level indicative of a voiding event.

In other cases, more complex characteristics may be used such as deviation of a signal from an amplitude or frequency range, e.g., exceeding an upper threshold or falling below a lower threshold. Other examples include signals have defined frequency characteristics that indicate a particular action, such as muscle movement, or nerve potentials or frequencies that indicate the onset of bladder contractions. In some cases, the sensor signal may have a well-established morphology with characteristic features that are generally present in the signal but vary in timing, frequency, amplitude, or interval, e.g., like an electrocardiogram (ECG) signal. Appropriate filter and amplifier circuitry, analog or digital, may be provided in the sensor or the processor to condition the signal so that such signal characteristics can be more specifically presented or isolated from extraneous information.

To identify a voiding event, neurostimulator 20 or external programmer 24 determines whether the signal output by each sensor matches its corresponding voiding signature. The signal output need not exactly match the corresponding voiding signature. Instead, a margin or difference threshold may be applied to indicate a voiding event if the sensor signal is within a given margin of the voiding signature. Again, the signature correlation may be as simple as comparing the sensor signal to a threshold or detecting a signal component in a particular frequency band or range. In more complex implementations, more detailed analysis of frequency and amplitude characteristics may be necessary to determine whether the sensor signal is sufficiently close to the voiding signature to indicate a voiding event.

As one example, a processor within neurostimulator 20 or external programmer 24 may generate a template signal corresponding to a voiding signature and apply a correlation technique. In some embodiments, a single sensor signal may be correlated with not just one, but multiple signal features, such as amplitude, frequency, time intervals, and the like. In addition, correlation values for the individual signal features may be weighted with coefficients to prioritize some features over other features. The correlation values for the individual features may be summed to produce an overall correlation value, which may be compared to a threshold value to identify a voiding event.

Using a digital signal processor (DSP), for example, the processor captures a series of samples of a sensor signal at a time that the patient or physician indicates that a voiding event is occurring. For example, the samples may be captured when the physician fills or empties the bladder during in-clinic analysis, or when the user enters affirmative input into external programmer 24 indicating that a voiding event is occurring or is about to occur In either case, the processor stores the sensor signal samples as a template signal that serves as a voiding signature. Then, for subsequent sensor signals, the processor performs a template-matching operation in which incoming samples from the sensor are compared to the stored voiding signature template.

The processor compares the samples for the newly acquired signal to the samples associated with the template and produces a correlation value. For example, the samples may be passed through a correlation window. The output of the correlation process may be correlation value expressed as a percentage or other value indicating the degree of similarity of the newly acquired signal to the template. Alternatively, the correlation value may be a binary output, i.e., a 1 for a positive correlation and a 0 for a negative correlation. In either case, the correlation value indicates the degree of similarity between the sensor signal and the template used as the voiding signature.

In other embodiments, the processor may quantify a severity or magnitude of the output and match the output to the voiding signature. The output may be correlated with multiple templates, or voiding signatures, to quantify the severity of the output. In this manner, the processor may utilize stored instructions or a lookup table to take different actions with regard to stimulation therapy based upon the determined severity of the output.

A high correlation value indicates substantial similarity between the sensor signal and the voiding signature, and indicates a high probability of a voiding event. If the correlation value exceeds a specified threshold, the processor concludes that the sensor signal is sufficiently similar to the template such that a match with the voiding signature can be declared. In this case, as a result of successful correlation, the processor indicates a voiding event, and directs whatever action may be necessary, such as activating or modifying electrical stimulation therapy, e.g., to permit voluntary voiding or prevent involuntary voiding.

If multiple sensors are used, the processor may apply multiple correlations between templates stored for respective sensors and the respective signals newly acquired from the sensors. Each template provides a voiding signature for a particular sensor. For example, the processor may store a pressure voiding signature, a nerve signal voiding signature, and a wetness voiding signature. Also, as mentioned previously, a negative voiding signature, e.g., from an accelerometer, may be provided as a cross-correlation to avoid false positive indications of voiding events. If the multiple sensors all present positive correlations, and there is no negative voiding signature, if applicable, the processor concludes that a voiding event is occurring. If only some of the sensors present positive correlations with their respective voiding signatures, the processor may indicate a voiding event if the number of sensors presenting positive correlations exceeds a predetermined threshold level.

Hence, each sensor may be associated with an individual correlation threshold to determine a positive correlation, and the group of sensors may be associated with another threshold for cross-correlation purposes to identify a voiding event. If only a single sensor or a small number of sensors yield a positive correlation with their respective voiding signature templates, the processor may still indicate the absence of a voiding event. On the other hand, if all sensors or a large number of sensors yield positive correlations, the processor indicates a voiding event. In some embodiments, the correlation values for the individual sensors may be assigned coefficients or weighting values to weight the correlation values from the sensors for consideration in the overall cross-correlation for identification of a voiding event.

FIG. 20 is a graph illustrating definition of a voiding signature as a classification map in a three-dimensional space defined by three different sensor signal values. As shown in FIG. 20, a classification map defines a region 212. If the coordinate values of the sensor signals on the three different axes, e.g., wetness, nerve signal potential, and pressure, map to a point within the three-dimensional region 212, then the three sensor signals are found to be indicative of a voiding event. Hence, in this case, points within three-dimensional region 212 are consistent with the voiding signature for the patient. The region 212 may be defining according to sensor signals obtained during an actual voiding event in the patient, or a simulated voiding event, e.g., by filling the bladder via a catheter by a physician to induce a bladder voiding event. The region 212 in the classification map essentially defines the voiding signature for which all sensors indicate that voiding is occurring.

The techniques described in this disclosure may be implemented in hardware, software, firmware or any combination thereof. For example, various aspects of the techniques may be implemented within one or more microprocessors, digital signal processors (DSPs), application specific integrated circuits (ASICs), field programmable logic arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry.

When implemented in software, the functionality ascribed to the systems and devices described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic media, optical media, or the like. The instructions are executed to support one or more aspects of the functionality described in this disclosure

Many embodiments of the invention have been described. Various modifications may be made without departing from the scope of the claims. For example, although the invention has been generally described in conjunction with implantable neurostimulation devices, a bladder sensor may also be used with other implantable medical devices, such as electrical muscle stimulation devices, functional electrical stimulation (FES) devices, and implantable drug delivery devices, each of which may be configured to treat incontinence or other conditions or disorders. These and other embodiments are within the scope of the following claims.

1. A method comprising:
receiving a sensor signal;
comparing the sensor signal to a voiding signature indicating an actual urinary voiding event in a patient; and
indicating a voiding event based on the comparison.

2. The method of claim 1, wherein the voiding signature specifies one or more characteristics of the sensor signal that indicate the actual voiding event.

3. The method of claim 1, wherein the voiding signature is defined based on one or more characteristics of the sensor signal during a natural voiding event.

4. The method of claim 1, wherein the voiding signature is defined based on one or more characteristics of the sensor signal during an induced voiding event.

5. The method of claim 1, further comprising delivering electrical stimulation therapy for urinary incontinence to the patient in response to the indication of the voiding event.

6. The method of claim 1, further comprising modifying one or more parameters associated with electrical stimulation therapy for urinary incontinence delivered to the patient in response to indication of the voiding event.

7. The method of claim 1, further comprising receiving a plurality of sensor signals, wherein the comparison includes comparing the sensor signals to respective voiding signatures indicating an actual urinary voiding event in a patient.

8. The method of claim 1, further comprising receiving a plurality of sensor signals, wherein the comparison includes determining whether the sensor signals map to a point within a region of a classification map corresponding to the voiding signature.

9. The method of claim 1, further comprising receiving a plurality of sensor signals, wherein the comparison includes comparing the sensor signals to respective voiding signatures indicating an actual urinary voiding event in a patient, and wherein the sensor signals indicate physiological conditions including two or more of nerve activity, bladder volume, bladder pressure, bladder impedance, sphincter pressure, and external wetness.

10. The method of claim 1, wherein the sensor signal indicates a physiological condition including at least one of nerve activity, bladder volume, bladder pressure, bladder impedance, sphincter pressure, and external wetness.

11. The method of claim 1, further comprising:
receiving a second sensor signal; and
determining whether the indication of a voiding event is false indication based on the second sensor signal.

12. The method of claim 1, further comprising storing the voiding event in a voiding log.

13. The method of claim 12, further comprising delivering electrical stimulation therapy for urinary incontinence to the patient in response to an analysis of the voiding log.

14. A system comprising:
a sensor that generates a sensor signal;
a processor that compares the sensor signal to a voiding signature indicating an actual urinary voiding event in a patient, and indicates a voiding event based on the comparison.

15. The system of claim 14, wherein the voiding signature specifies one or more characteristics of the sensor signal that indicate the actual voiding event.

16. The system of claim 14, wherein the voiding signature is defined based on one or more characteristics of the sensor signal during a natural voiding event.

17. The system of claim 14, wherein the voiding signature is defined based on one or more characteristics of the sensor signal during an induced voiding event.

18. The system of claim 14, further comprising an electrical stimulator that delivers electrical stimulation therapy for urinary incontinence to the patient in response to the indication of the voiding event.

19. The system of claim 14, further comprising an electrical stimulator that delivers electrical stimulation therapy for urinary incontinence to the patient, wherein the processor modifies one or more parameters associated with the electrical stimulation therapy in response to the indication of the voiding event.

20. The system of claim 14, further comprising a plurality of sensors that generate a plurality of sensor signals, wherein the processor compares the sensor signals to respective voiding signatures indicating an actual urinary voiding event in a patient.

21. The system of claim 14, further comprising a plurality of sensors that generate a plurality of sensor signals, wherein the comparison by the processor includes determining whether the sensor signals map to a point within a region of a classification map corresponding to the voiding signature.

22. The system of claim 14, further comprising a plurality of sensors that generate a plurality of sensor signals, wherein the processor compares the sensor signals to respective voiding signatures indicating an actual urinary voiding event in a patient, and wherein the sensor signals indicate physiological conditions including two or more of nerve activity, bladder volume, bladder pressure, bladder impedance, sphincter pressure, and external wetness.

23. The system of claim 14, wherein the sensor signal indicates a physiological condition including at least one of nerve activity, bladder volume, bladder pressure, bladder impedance, sphincter pressure, and external wetness.

24. The system of claim 14, further comprising a second sensor that generates a second sensor signal, and the processor determines whether the indication of a voiding event is false indication based on the second sensor signal.

25. The system of claim 14, further comprising an electrical stimulator that delivers electrical stimulation therapy for urinary incontinence to the patient in response to the indication of the voiding event, wherein the processor resides within the stimulator.

26. The system of claim 14, further comprising an electrical stimulator that delivers electrical stimulation therapy for urinary incontinence to the patient in response to the indication of the voiding event, and an external device that communicates with the electrical stimulator, wherein the processor resides within the external device.

27. A computer-readable medium comprising instructions to cause a processor to:
receive a sensor signal;
compare the sensor signal to a voiding signature indicating an actual urinary voiding event in a patient; and
indicate a voiding event based on the comparison.

28. The computer-readable medium of claim 27, wherein the voiding signature specifies one or more characteristics of the sensor signal that indicate the actual voiding event.

29. The computer-readable medium of claim 27, wherein the voiding signature is defined based on one or more characteristics of the sensor signal during a natural voiding event, or one or more characteristics of the sensor signal during an induced voiding event.

30. The computer-readable medium of claim 27, wherein the instructions cause the processor to modify one or more parameters associated with electrical stimulation therapy for urinary incontinence delivered to the patient in response to indication of the voiding event.

31. The computer-readable medium of claim 27, wherein the instructions cause the processor to receive a plurality of sensor signals, wherein the comparison includes comparing the sensor signals to respective voiding signatures indicating an actual urinary voiding event in a patient.

32. The computer-readable medium of claim 27, wherein the instructions cause the processor to receive a plurality of sensor signals, wherein the comparison includes determining whether the sensor signals map to a point within a region of a classification map corresponding to the voiding signature.

33. The computer-readable medium of claim 27, wherein the instructions cause the processor to receive a plurality of sensor signals, wherein the comparison includes comparing the sensor signals to respective voiding signatures indicating an actual urinary voiding event in a patient, and wherein the sensor signals indicate physiological conditions including two or more of nerve activity, bladder volume, bladder pressure, bladder impedance, sphincter pressure, and external wetness.

34. The computer-readable medium of claim 27, wherein the instructions cause the processor to:
receive a second sensor signal; and
determine whether the indication of a voiding event is false indication based on the second sensor signal.

35. A system comprising:
means for receiving a sensor signal;
means for comparing the sensor signal to a voiding signature indicating an actual urinary voiding event in a patient; and
means for indicating a voiding event based on the comparison.

36. The system of claim 35, wherein the voiding signature specifies one or more characteristics of the sensor signal that indicate the actual voiding event.

37. The system of claim 35, wherein the voiding signature is defined based on one or more characteristics of the sensor signal during a natural voiding event or one or more characteristics of the sensor signal during an induced voiding event.
